(19) United States
(12) Patent Application Publication
(10) Pub. No.: US 2006/0073479 A1
(43) Pub. Date: Apr. 6, 2006

(54) Single nucleotide polymorphisms and combinations thereof predictive for paclitaxel responsiveness

(76) Inventor: Tony N Frudakis, Bradenton, FL (US)

Correspondence Address: DLA PIPER RUDNICK GRAY CARY US, LLP, 4365 EXECUTIVE DRIVE, SUITE 1100, SAN DIEGO, CA, 92121-2133, US

(21) Appl. No.: 10/496,605

(22) PCT Filed: Nov. 26, 2002
(86) PCT No.: PCT/US02/38345

§ 371 Date: Mar. 3, 2005

Related U.S. Application Data
(60) Provisional application No. 60/334,310, filed on Nov. 28, 2001.
(60) Provisional application No. 60/410,363, filed on Sep. 11, 2002.

(51) Int. Cl. C12Q 1/68; C07H 21/04

(52) U.S. Cl. 435/6; 536/24.3

(57) ABSTRACT

Single nucleotide polymorphisms (SNPs) and combinations of SNPs that allow an inference as to whether a cancer patient is likely to respond or not respond to paclitaxel (Taxol®) are provided. Also provided are methods of determining a whether a cancer patient should be treated with paclitaxel.

### BACKGROUND OF THE INVENTION

1. Field of the Invention

The invention relates generally to genetic markers useful for determining the responsiveness of a cancer patient to a treatment, and more specifically to single nucleotide polymorphisms (SNPs) and combinations of SNPs that allow an inference as to whether treatment with paclitaxel is likely to result in a therapeutic benefit to a cancer patient, and to methods of determining a course of treatment for a cancer patient based on such SNPs and combinations thereof.

2. Background Information

Cancer is a leading cause of morbidity and mortality in most developed countries. Although some cancer have been linked directly to specific causes, for example, lung cancer in smokers, the cause of most cancers is not clearly defined. As such, few if any specific methods for preventing cancer are known and, therefore, much work has focused on improving methods of treating cancer in patients.

Cancers generally are treated by surgery, chemotherapy, and radiation therapy, either alone or in combination. Surgical procedures have been improved, for example, by using computer assisted techniques, including high resolution imaging techniques, and microsurgery to more selectively excise tumor tissue while sparing normal tissue. In contrast to the selectivity of surgery, chemotherapy acts more systemically and is particularly useful for treating disseminated disease, including metastases. Unfortunately, the lack of selectivity of chemotherapy often results in substantial damage to otherwise healthy tissues, including rapidly dividing cells such as bone marrow and epithelial cells.

The potential effectiveness of chemotherapy also can be limited due, for example, to selection of tumor cells that are resistant to the chemotherapeutic agent. Xenobiotic metabolism genes encode polypeptides that act to detoxify foreign compounds present in the body. While these genes evolved to allow humans to degrade and excrete harmful chemicals such as tannins and alkaloids, which are present in many foods and from which many drugs are derived, they also can act to reduce the toxic effect of chemotherapeutic agents.

The common anti-cancer drug, paclitaxel (Taxol®), is an example of a chemotherapeutic agent that is metabolized in the human body, in this case by members of the cytochrome P450 family members, CYP2C8 and, to a lesser extent, CYP3A4. Paclitaxel is isolated from the Pacific yew tree, and belongs to the group of medicines called antineoplastics. Paclitaxel has proven useful to treat cancer of the ovaries, breast, certain types of lung cancer, and a cancer of the skin and mucous membranes that occurs in patients with acquired immunodeficiency syndrome (AIDS). While paclitaxel can provide a significant therapeutic benefit to many patients, about one-third of the patients treated with paclitaxel are refractory to its beneficial effect. As such, paclitaxel treatment is initiated in a large number of patients that, ultimately, are not going to respond to the therapy. Since it takes time for a beneficial effect of paclitaxel treatment to become observable, significant time can be lost treating patients that otherwise could be treated using alternative modalities.

Since paclitaxel is metabolized in the body, it has been speculated that there may a genetic basis for the differences in responsiveness of cancer patients to paclitaxel treatment, for example, a change that result in over-expression of a cytochrome P450 protein or a change that results expression of a mutant cytochrome P450 having greater metabolizing activity. However, no correlation between a genetic change in a cytochrome P450 gene and paclitaxel responsiveness has been described. As such, there is no way to predict with any certainty whether a cancer patient is likely to respond to paclitaxel treatment. Thus, a need exists to identify genetic markers that are predictive as to the responsiveness of a patient to a paclitaxel therapy. The present invention satisfies this need and provides additional advantages.

### SUMMARY OF THE INVENTION

The present invention is based, in part, on the identification of single nucleotide polymorphisms (SNPs) that, alone or in combination, allow an inference to be drawn as to whether a cancer patient will respond (or not respond) to paclitaxel. Accordingly, the invention provides oligonucleotide probes and primers useful for detecting nucleotide occurrences at the positions of SNPs that allow an inference as to paclitaxel responsiveness, including combinations of such probes and primers; and also provides methods of inferring whether a subject is likely to be a responder or a non-responder to paclitaxel.

The present invention relates to a method for inferring responsiveness of a subject to paclitaxel treatment from a nucleic acid sample of the subject. In one embodiment, a method of the invention can be performed by detecting, in the nucleic acid sample, the nucleotide occurrence of a SNP associated with paclitaxel responsiveness. As disclosed herein, SNPs associated with paclitaxel responsiveness include nucleotides of a cytochrome P450 (CYP2C8) gene, including a nucleotide corresponding to nucleotide 83 of SEQ ID NO:1, nucleotide 251 of SEQ ID NO:2; nucleotide 181 of SEQ ID NO:15, or nucleotide 75 of SEQ ID NO:35; nucleotides of a CYP3A4 gene, including a nucleotide corresponding to nucleotide 401 of SEQ ID NO:3, nucleotide 437 of SEQ ID NO:4; nucleotide 151 of SEQ ID NO:16, or nucleotide 1466 of SEQ ID NO:34; nucleotides of an esterase D (ESD) gene, including a nucleotide corresponding to nucleotide 702 of SEQ ID NO:5, or nucleotide 201 of SEQ ID NO:6; nucleotides of a glutathione S-transferase (GSTM1) gene, including a nucleotide corresponding to nucleotide 201 of SEQ ID NO:7, or nucleotide 191 of SEQ ID NO:8; nucleotides of a CYP3A7 gene, including a nucleotide corresponding to nucleotide 401 of SEQ ID NO:9, nucleotide 541 of SEQ ID NO:10, nucleotide 151 of SEQ ID NO:17, or nucleotide 201 of SEQ ID NO:21; nucleotides of a monoamine oxidase (MAOB) gene, including a nucleotide corresponding to nucleotide 501 of SEQ ID NO:11, or nucleotide 60 of SEQ ID NO:12; nucleotides of a CYP3A5 gene, including a nucleotide corresponding to nucleotide 201 of SEQ ID NO:13, or nucleotide 101 of SEQ ID NO:14; nucleotides of an agouti signaling protein (ASIP) gene, including a nucleotide corresponding to nucleotide 201 of SEQ ID NO:22, nucleotide 26 of SEQ ID NO:30, or nucleotide 101 of SEQ ID NO:32; nucleotides of a tubulin (TUBB) gene, including a nucleotide corresponding to nucleotide 61 of SEQ ID NO:18; nucleotides of a CYP2C9 gene, including a nucleotide corresponding to nucleotide 122 of SEQ ID NO:19; nucleotides of a tyrosinase-related protein (TYRP) gene, including a nucleotide corresponding to nucleotide 592 of SEQ ID NO:20; nucleotides of a CYP2D6 gene, including a nucleotide corresponding to nucleotide 201 of SEQ ID NO:23; nucleotides of a membrane-associated transporter protein (AIM; also called MATP) gene, including nucleotide 201 of SEQ ID NO24; nucleotides of a GSTT gene, including a nucleotide corresponding to nucleotide 61 of SEQ ID NO:25; nucleotides of a dopachrome tautomerase (DCT) gene, including a nucleotide corresponding to nucleotide 201 of SEQ ID NO:26, or nucleotide 61 of SEQ ID NO:27; nucleotides of an oculocutaneous albinism (OCA) gene, including a nucleotide corresponding to nucleotide 135 of SEQ ID NO:28; nucleotides of a CYP4B gene, including a nucleotide corresponding to nucleotide 123 of SEQ ID NO:29; nucleotides of a cytochrome P450 oxidoreductase (POR) gene, including a nucleotide corresponding to nucleotide 61 of SEQ ID NO:31; nucleotides of a ras-like GTP binding protein (RAB; also called RAB27A) gene, including a nucleotide corresponding to nucleotide 201 of SEQ ID NO:33; or a combination of SNPs including nucleotides as set forth above, wherein the SNP or combination of SNPs indicates that the subject is a paclitaxel responder or that the subject is a paclitaxel non-responder.

In one aspect of this embodiment, SNPs associated with paclitaxel responsiveness that are detected include nucleotides of a CYP2C8 gene, including a nucleotide corresponding to nucleotide 83 of SEQ ID NO:1, nucleotide 251 of SEQ ID NO:2; nucleotide 181 of SEQ ID NO:15, or nucleotide 75 of SEQ ID NO:35; nucleotides of a CYP3A4 gene, including a nucleotide corresponding to nucleotide 401 of SEQ ID NO:3, nucleotide 437 of SEQ ID NO:4; nucleotide 151 of SEQ ID NO:16, or nucleotide 1466 of SEQ ID NO:34; nucleotides of an ESD gene, including a nucleotide corresponding to nucleotide 702 of SEQ ID NO:5, or nucleotide 201 of SEQ ID NO:6; nucleotides of a GSTM1 gene, including a nucleotide corresponding to nucleotide 201 of SEQ ID NO:7, or nucleotide 191 of SEQ ID NO:8; nucleotides of a CYP3A7 gene, including a nucleotide corresponding to nucleotide 401 of SEQ ID NO:9, nucleotide 541 of SEQ ID NO:10, nucleotide 151 of SEQ ID NO:17, or nucleotide 201 of SEQ ID NO:21; nucleotides of an MAOB gene, including a nucleotide corresponding to nucleotide 501 of SEQ ID NO:11, or nucleotide 60 of SEQ ID NO:12; nucleotides of a CYP3A5 gene, including a nucleotide corresponding to nucleotide 201 of SEQ ID NO:13.

In another aspect of this embodiment, SNPs associated with paclitaxel responsiveness that are detected include nucleotide 83 of SEQ ID NO:1, nucleotide 251 of SEQ ID NO:2, nucleotide 401 of SEQ ID NO:3, nucleotide 702 of SEQ ID NO:5, nucleotide 201 of SEQ ID NO:6, nucleotide 201 of SEQ ID NO:7, nucleotide 191 of SEQ ID NO:8, nucleotide 401 of SEQ ID NO:9, nucleotide 541 of SEQ ID NO:10, nucleotide 501 of SEQ ID NO:1, nucleotide 60 of SEQ ID NO:12, nucleotide 201 of SEQ ID NO:13, nucleotide 101 of SEQ ID NO:14, nucleotide 181 of SEQ ID NO:15, nucleotide 151 of SEQ ID NO:16, nucleotide 151 of SEQ ID NO:17, nucleotide 75 of SEQ ID NO:35, or a combination thereof.

In still another aspect of this embodiment, SNPs associated with paclitaxel responsiveness that are detected include nucleotide 83 of SEQ ID NO:1, nucleotide 251 of SEQ ID NO:2, nucleotide 401 of SEQ ID NO:3, nucleotide 437 of SEQ ID NO:4, nucleotide 702 of SEQ ID NO:5, nucleotide 201 of SEQ ID NO:6, nucleotide 201 of SEQ ID NO:7, nucleotide 191 of SEQ ID NO:8, nucleotide 401 of SEQ ID NO:9, nucleotide 541 of SEQ ID NO:10, nucleotide 501 of SEQ ID NO:11, nucleotide 60 of SEQ ID NO:12, nucleotide 201 of SEQ ID NO: 13, nucleotide 101 of SEQ ID NO:14, nucleotide 181 of SEQ ID NO:15, nucleotide 151 of SEQ ID NO:16, nucleotide 151 of SEQ ID NO:17, nucleotide 61 of SEQ ID NO:18, nucleotide 122 of SEQ ID NO:19, nucleotide 26 of SEQ ID NO:30, nucleotide 75 of SEQ ID NO:35, or a combination thereof.

According to a method of the invention, an inference can be made that subject is likely to be a paclitaxel responder by detecting, for example, that the nucleotide occurrence at nucleotide 83 of SEQ ID NO:1 is A; nucleotide 251 of SEQ ID NO:2 is G or A; nucleotide 401 of SEQ ID NO:3 is C; nucleotide 437 of SEQ ID NO:4 is T; nucleotide 702 of SEQ ID NO:5 is C or T; nucleotide 201 of SEQ ID NO:6 is C; nucleotide 201 of SEQ ID NO:7 is C; nucleotide 191 of SEQ ID NO:8 is C or T; nucleotide 401 of SEQ ID NO:9 is T; nucleotide 541 of SEQ ID NO:10 is G; nucleotide 501 of SEQ ID NO:11 is T; nucleotide 60 of SEQ ID NO:12 is T; nucleotide 201 of SEQ ID NO:13 is G; nucleotide 101 of SEQ ID NO:14 is T; nucleotide 181 of SEQ ID NO:15 is G; nucleotide 151 of SEQ ID NO:16 is C; nucleotide 151 of SEQ ID NO:17 is C; nucleotide 61 of SEQ ID NO:18 is G; nucleotide 122 of SEQ ID NO:19 is A; nucleotide 26 of SEQ ID NO:30 is A; nucleotide 75 of SEQ ID NO:35 is G; or a combination thereof.

In addition, an inference can be made that subject is likely to be a paclitaxel non-responder by detecting, for example, that the nucleotide occurrence at nucleotide 83 of SEQ ID NO:1 is G; nucleotide 251 of SEQ ID NO:2 is G or A; nucleotide 401 of SEQ ID NO:3 is T; nucleotide 437 of SEQ ID NO:4 is G; nucleotide 702 of SEQ ID NO:5 is C or T; nucleotide 201 of SEQ ID NO:6 is G; nucleotide 201 of SEQ ID NO:7 is T; nucleotide 191 of SEQ ID NO:8 is C or T; nucleotide 401 of SEQ ID NO:9 is C; nucleotide 541 of SEQ ID NO:10 is C; nucleotide 501 of SEQ ID NO:11 is C; nucleotide 60 of SEQ ID NO:12 is C; nucleotide 201 of SEQ ID NO:13 is A; nucleotide 101 of SEQ ID NO:14 is C; nucleotide 181 of SEQ ID NO:15 is A; nucleotide 151 of SEQ ID NO:16 is T; nucleotide 151 of SEQ ID NO:17 is T; nucleotide 61 of SEQ ID NO:18 is A; nucleotide 122 of SEQ ID NO:19 is T; nucleotide 26 of SEQ ID NO:30 is G; nucleotide 75 of SEQ ID NO:35 is A; or a combination thereof. As disclosed herein, combinations of such gene nucleotide sequences as recited above also are provided.

In another embodiment, a method for inferring responsiveness of a subject to paclitaxel treatment from a nucleic acid sample of the subject is performed by detecting, in the nucleic acid sample, a haplotype allele associated with paclitaxel responsiveness. Such an inference can be made by detecting, for example, a haplotype allele that includes nucleotides of a CYP2C8 gene, including nucleotides corresponding to at least two of nucleotide 83 of SEQ ID NO:1, nucleotide 251 of SEQ ID NO:2; nucleotide 181 of SEQ ID NO:15, and nucleotide 75 of SEQ ID NO:35; nucleotides of a CYP3A4 gene, including nucleotides corresponding to at least two of nucleotide 401 of SEQ ID NO:3, nucleotide 437 of SEQ ID NO:4; nucleotide 151 of SEQ ID NO:16, and nucleotide 1466 of SEQ ID NO:34; nucleotides of an ESD gene, including nucleotides corresponding to at least nucleotide 702 of SEQ ID NO:5, and nucleotide 201 of SEQ ID NO:6; nucleotides of a GSTM1 gene, including nucleotides corresponding to at least nucleotide 201 of SEQ ID NO:7, and nucleotide 191 of SEQ ID NO:8; nucleotides of a CYP3A7 gene, including nucleotides corresponding to at least two of nucleotide 401 of SEQ ID NO:9, nucleotide 541 of SEQ ID NO:10, nucleotide 151 of SEQ ID NO:17, and nucleotide 201 of SEQ ID NO:21; nucleotides of a MAOB gene, including nucleotides corresponding to at least nucleotide 501 of SEQ ID NO:1, and nucleotide 60 of SEQ ID NO:12; nucleotides of a CYP3A5 gene, including nucleotides corresponding to at least nucleotide 201 of SEQ ID NO:13, and nucleotide 101 of SEQ ID NO:14; nucleotides of an ASIP gene, including nucleotides corresponding to at least two of nucleotide 201 of SEQ ID NO:22, nucleotide 26 of SEQ ID NO:30, and nucleotide 101 of SEQ ID NO:32; nucleotides of a TUBB gene, including nucleotides corresponding to nucleotide 61 of SEQ ID NO:18, and at least a second SNP of a TUBB gene, wherein the SNP is associated with paclitaxel responsiveness; nucleotides of a CYP2C9 gene, including nucleotides corresponding to nucleotide 122 of SEQ ID NO:19, and at least a second SNP of a CYP2C9 gene, wherein the SNP is associated with paclitaxel responsiveness; nucleotides of a TYRP gene, including nucleotides corresponding to nucleotide 592 of SEQ ID NO:20, and at least a second SNP of a TYRP gene, wherein the SNP is associated with paclitaxel responsiveness; nucleotides of a CYP2D6 gene, including nucleotides corresponding to nucleotide 201 of SEQ ID NO:23, and at least a second SNP of a CYP2D6 gene, wherein the SNP is associated with paclitaxel responsiveness; nucleotides of an AIM gene, including nucleotides corresponding to nucleotide 201 of SEQ ID NO₂₄, and at least a second SNP of an AIM gene, wherein the SNP is associated with paclitaxel responsiveness; nucleotides of a GSTT gene, including nucleotides corresponding to nucleotide 61 of SEQ ID NO:25, and at least a second SNP of a GST gene, wherein the SNP is associated with paclitaxel responsiveness; nucleotides of a DCT gene, including nucleotides corresponding to at least nucleotide 201 of SEQ ID NO:26, and nucleotide 61 of SEQ ID NO:27; nucleotides of an OCA gene, including nucleotides corresponding to nucleotide 135 of SEQ ID NO:28, and at least a second SNP of a OCA gene, wherein the SNP is associated with paclitaxel responsiveness; nucleotides of a CYP4B gene, including nucleotides corresponding to nucleotide 123 of SEQ ID NO:29, and at least a second SNP of a CYP4B gene, wherein the SNP is associated with paclitaxel responsiveness; nucleotides of a POR gene, including nucleotide 61 of SEQ ID NO:31, and at least a second SNP of a POR gene, wherein the SNP is associated with paclitaxel responsiveness; nucleotides of an RAB gene, including nucleotides corresponding to nucleotide 201 of SEQ ID NO:33, and at least a second SNP of a RAB gene, wherein the SNP is associated with paclitaxel responsiveness; or a combination of haplotype alleles as set forth above, wherein the haplotype allele or combination of haplotype alleles indicates that the subject is a paclitaxel responder or that the subject is a paclitaxel non-responder.

In one aspect of this embodiment, an inference as to paclitaxel responsiveness can be made by detecting a haplotype allele that includes nucleotides of a CYP2C8 gene, which include nucleotide 83 of SEQ ID NO:1, and nucleotide 251 of SEQ ID NO:2; nucleotides of a CYP3A4 gene, which include nucleotide 401 of SEQ ID NO:3, and nucleotide 437 of SEQ ID NO:4; nucleotides of an ESD gene, which include nucleotide 702 of SEQ ID NO:5, and nucleotide 201 of SEQ ID NO:6; nucleotides of a GSTM1 gene, which include nucleotide 201 of SEQ ID NO:7, and nucleotide 191 of SEQ ID NO:8; nucleotides of a CYP3A7 gene, which include nucleotide 401 of SEQ ID NO:9, and nucleotide 541 of SEQ ID NO:10; nucleotides of a MAOB gene, which include nucleotide 501 of SEQ ID NO:11, and nucleotide 60 of SEQ ID NO:12; nucleotides of a CYP3A5 gene, which include nucleotide 201 of SEQ ID NO:13, and nucleotide 101 of SEQ ID NO:14; or a combination of such haplotype alleles. For example, an inference as to paclitaxel responsiveness can be made by detecting a CYP2C8 gene haplotype allele that includes GG or GA; a CYP3A4 gene haplotype allele that includes CT; an ESD gene haplotype allele that includes TC or CC; a GSTM1 gene haplotype allele that includes TC; a CYP3A7 gene haplotype allele that includes TG; an MAOB gene haplotype allele that includes CC; a CYP3A5 gene haplotype allele that includes GT, or a combination of such haplotype alleles.

According to a method of the invention, an inference can be made that subject is likely to be a paclitaxel responder by detecting, for example, that the haplotype allele or combination of haplotype alleles in the subject includes CYP2C8 gene haplotype alleles other than GG or GA; a CYP3A4 gene haplotype allele CT; an ESD gene haplotype allele TC or CC; GSTM1 gene haplotype alleles other than TC; a CYP3A7 gene haplotype allele TG; MAOB gene haplotype alleles other than CC; a CYP3A5 gene haplotype allele GT; or a combination thereof. The method can further include determining both haplotype alleles of a subject, including, for example, CYP2C8 gene diploid haplotype alleles other than GG/NN or GA/NN; CYP3A4 gene diploid haplotype alleles CT/CT; ESD gene diploid haplotype alleles TC/TC or CC/CC; GSTM1 gene diploid haplotype alleles other than TC/NN; CYP3A7 gene diploid haplotype alleles TG/TG; MAOB gene diploid haplotype alleles other than CC/NN; CYP3A5 gene diploid haplotype alleles GT/GT; or a combination thereof, the detection of which allows an inference that the subject likely is a paclitaxel responder. In addition, an inference can be made that a subject is a paclitaxel non-responder by detecting that the subject has CYP3A4 gene haplotype alleles other than CT, including by further determining that the subject has CYP3A4 gene diploid haplotype alleles other than CT/CT.

In yet another embodiment, a method for inferring paclitaxel responsiveness of a subject from a nucleic acid sample of the subject is performed by detecting, in the nucleic acid sample, diploid haplotype alleles associated with paclitaxel non-response. The diploid haplotype alleles can include, for example, nucleotides of a CYP2C8 gene, including nucleotides corresponding to nucleotide 83 of SEQ ID NO:1, and nucleotide 251 of SEQ ID NO:2; nucleotides of a CYP3A4 gene, including nucleotides corresponding to nucleotide 401 of SEQ ID NO:3, and nucleotide 437 of SEQ ID NO:4; nucleotides of an ESD gene, including e nucleotides corresponding to nucleotide 702 of SEQ ID NO:5, and nucleotide 201 of SEQ ID NO:6; nucleotides of a GSTM1 gene, which comprise nucleotides corresponding to nucleotide 201 of SEQ ID NO:7, and nucleotide 191 of SEQ ID NO:8; nucleotides of a CYP3A7 gene, including e nucleotides corresponding to nucleotide 401 of SEQ ID NO:9, and nucleotide 541 of SEQ ID NO:10; nucleotides of a MAOB gene, including nucleotides corresponding to nucleotide 501 of SEQ ID NO:11, and nucleotide 60 of SEQ ID NO:12; nucleotides of a CYP3A5 gene, including nucleotides corresponding to nucleotide 201 of SEQ ID NO:13, and nucleotide 101 of SEQ ID NO:14; or a combination of such diploid haplotype alleles. For example, an inference can be made that a subject is a non-responder with respect to paclitaxel by detecting the CYP2C8 gene diploid haplotype alleles GG/NN or GA/NN; the CYP3A4 gene diploid haplotype alleles CT/CT; the ESD gene diploid haplotype alleles TC/NN or CC/NN; the GSTM1 gene diploid haplotype alleles TC/NN; the CYP3A7 gene diploid haplotype alleles TG/TG; the MAOB gene diploid haplotype alleles CC/NN; or the CYP3A5 gene diploid haplotype alleles GT/GT; or a combination thereof.

In one aspect of this embodiment, an inference can be made that a subject is likely to be a responder to paclitaxel by detecting diploid haplotype alleles, or combination thereof, including, for example, CYP2C8 gene diploid haplotype alleles other than GG/NN or GA/NN; the CYP3A4 gene diploid haplotype alleles CT/CT; the ESD gene diploid haplotype alleles TC/TC or CC/CC; GSTM1 gene diploid haplotype alleles other than TC/NN; CYP3A7 gene diploid haplotype alleles TG/TG; MAOB gene diploid haplotype alleles other than CC/NN; the CYP3A5 gene diploid haplotype alleles GT/GT. In another aspect of this embodiment, an inference can be made that a subject is a paclitaxel non-responder by detecting, for example, that the subject has CYP3A4 gene diploid haplotype alleles other than CT/CT. As disclosed herein, combinations of nucleotide sequences comprising the above recited haplotype alleles also are provided.

The present invention also relates to an isolated nucleic acid molecule, which includes a SNP associated with paclitaxel responsiveness. Such an isolated nucleic acid molecule includes at least 15 contiguous nucleotides (e.g., 15, 18, 21, 25, 30, 40, 50, or more) of a polynucleotide as set forth in SEQ ID NO:1, including at least nucleotide 83 of SEQ ID NO:1, wherein nucleotide 83 is G; SEQ ID NO:3, including at least nucleotide 401 of SEQ ID NO:3, wherein nucleotide 401 is T; SEQ ID NO:4, including at least nucleotide 437 of SEQ ID NO:4, wherein nucleotide 437 is G; SEQ ID NO:8, including at least nucleotide 191 of SEQ ID NO:8, wherein nucleotide 191 is T, or a polynucleotide complementary to such an isolated nucleic acid molecule. The isolated nucleic acid molecule can be a polyribonucleotide (RNA) or a polydeoxyribonucleotide (DNA), and can be single stranded or double stranded, including, for example, a DNA/RNA hybrid.

The invention further relates to a kit containing an isolated nucleic acid molecule of the invention. In addition, the kit can further include an oligonucleotide that selectively hybridizes to the nucleic acid molecule of the kit, particularly an oligonucleotide that selectively hybridizes at or near nucleotide 83 of SEQ ID NO:1; nucleotide 401 of SEQ ID NO:3; nucleotide 437 of SEQ ID NO:4, or nucleotide 191 of SEQ ID NO:8.

The present invention also relates to plurality of isolated nucleic acid molecules, wherein the plurality includes at least one isolated nucleic acid molecule of the invention, for example, two, three, or all four of the nucleic acid molecules of the invention. The plurality of isolated nucleic acid molecules, in addition to at least one nucleic acid molecule of the invention, can further contain a corresponding nucleic acid molecule representing the other variant of the at least one isolated nucleic acid molecule. For example, the kit can contain a nucleic acid molecule of at least 15 contiguous nucleotides of a polynucleotide as set forth in SEQ ID NO:1, including at least nucleotide 83 of SEQ ID NO:1, wherein nucleotide 83 is A; SEQ ID NO:3, including at least nucleotide 401 of SEQ ID NO:3, wherein nucleotide 401 is C; SEQ ID NO:4, including at least nucleotide 437 of SEQ ID NO:4, wherein nucleotide 437 is T; or SEQ ID NO:8, including at least nucleotide 191 of SEQ ID NO:8, wherein nucleotide 191 is C, or a polynucleotide complementary thereto.

A kit containing a plurality of isolated nucleic acid molecules also can contain one or more nucleic acid molecules of at least 15 contiguous nucleotides of a polynucleotide including nucleotide 251 of SEQ ID NO:2; nucleotide 702 of SEQ ID NO:5; nucleotide 201 of SEQ ID NO:6; nucleotide 201 of SEQ ID NO:7; nucleotide 191 of SEQ ID NO:8; nucleotide 401 of SEQ ID NO:9; nucleotide 541 of SEQ ID NO:10; nucleotide 501 of SEQ ID NO:11; nucleotide 60 of SEQ ID NO:12; nucleotide 201 of SEQ ID NO:13; nucleotide 101 of SEQ ID NO:14; nucleotide 181 of SEQ ID NO:15; nucleotide 151 of SEQ ID NO:16; nucleotide 151 of SEQ ID NO:17; nucleotide 61 of SEQ ID NO:18; nucleotide 122 of SEQ ID NO:19; nucleotide 592 of SEQ ID NO:20; nucleotide 201 of SEQ ID NO:21; nucleotide 201 of SEQ ID NO:22; nucleotide 201 of SEQ D NO:23; nucleotide 201 of SEQ ID NO:24; nucleotide 61 of SEQ ID NO:25; nucleotide 201 of SEQ ID NO:26, or nucleotide 61 of SEQ ID NO:27; nucleotide 135 of SEQ ID NO:28; nucleotide 123 of SEQ ID NO:29; nucleotide 26 of SEQ ID NO:30; nucleotide 61 of SEQ ID NO:31; nucleotide 101 of SEQ ID NO:32; nucleotide 201 of SEQ ID NO:33, nucleotide 1466 of SEQ ID NO:34; or nucleotide 75 of SEQ ID NO:35, or a nucleotide sequence complementary to any of the foregoing.

The present invention also relates to a kit containing such a plurality of nucleic acid molecules, wherein the kit can further contain one or more oligonucleotides that selectively hybridize at or near the specified nucleotide of each of the nucleic acid molecules of the plurality. Such oligonucleotides are useful, for example, for identifying the present of a nucleic acid molecule containing the polymorphic nucleotide in a sample. Accordingly, the invention also provides oligonucleotide probes and/or primers that selectively hybridize at or near, respectively, a SNP position as disclosed herein.

The present invention further relates to a plurality of oligonucleotides, comprising at least one oligonucleotide that selectively hybridizes to a nucleotide sequence as set forth in SEQ ID NO:1, including nucleotide 83 of SEQ ID NO:1, wherein nucleotide 83 is G; SEQ ID NO:3, including nucleotide 401 of SEQ ID NO:3, wherein nucleotide 401 is T; SEQ ID NO:4, including nucleotide 437 of SEQ ID NO:4, wherein nucleotide 437 is G; or SEQ ID NO:8, including nucleotide 191 of SEQ ID NO:8, wherein nucleotide 191 is T, or to a nucleotide sequence complementary thereto. The plurality can contain one of the above oligonucleotides and a second oligonucleotide of interest, or can contain two, three or four of the above oligonucleotides and, if desired, one or more other oligonucleotides of interest, such oligonucleotides being useful as probes, primers, or the like. For example, the plurality of oligonucleotides can include at least one oligonucleotide that selectively hybridizes at or near a nucleotide sequence as set forth in SEQ ID NO:1, said nucleotide sequence comprising nucleotide 83 of SEQ ID NO:1, wherein nucleotide 83 is A; SEQ ID NO:3, said nucleotide sequence comprising nucleotide 401 of SEQ ID NO:3, wherein nucleotide 401 is C; SEQ BD NO:4, said nucleotide sequence comprising nucleotide 437 of SEQ ID NO:4, wherein nucleotide 437 is T; or SEQ ID NO:8, said nucleotide sequence comprising nucleotide 191 of SEQ ID NO:8, wherein nucleotide 191 is C, or a nucleotide sequence complementary thereto, such a plurality being useful, for example, to identify in a nucleic acid sample one or both polymorphic variants corresponding to the specified SNP position (e.g., the SNP at a position in a genomic DNA sample corresponding to nucleotide 83 of SEQ ID NO:1, which can be A or G).

The plurality of oligonucleotides also can include, for example, at least one oligonucleotide that selectively hybridizes at or near nucleotide 251 of SEQ ID NO:2; nucleotide 702 of SEQ ID NO:5; nucleotide 201 of SEQ ID NO:6; nucleotide 201 of SEQ ID NO:7; nucleotide 191 of SEQ ID NO:8; nucleotide 401 of SEQ ID NO:9; nucleotide 541 of SEQ ID NO:10; nucleotide 501 of SEQ ID NO:11; nucleotide 60 of SEQ ID NO:12; nucleotide 201 of SEQ ID NO:13; nucleotide 101 of SEQ ID NO:14; nucleotide 181 of SEQ ID NO:15; nucleotide 151 of SEQ ID NO:16; nucleotide 151 of SEQ ID NO:17; nucleotide 61 of SEQ ID NO:18; nucleotide 122 of SEQ ID NO:19; nucleotide 592 of SEQ ID NO:20; nucleotide 201 of SEQ ID NO:21; nucleotide 201 of SEQ ID NO:22; nucleotide 201 of SEQ ID NO:23; nucleotide 201 of SEQ ID NO:24; nucleotide 61 of SEQ ID NO:25; nucleotide 201 of SEQ ID NO:26, or nucleotide 61 of SEQ ID NO:27; nucleotide 135 of SEQ ID NO:28; nucleotide 123 of SEQ ID NO:29; nucleotide 26 of SEQ ID NO:30; nucleotide 61 of SEQ ID NO:31; nucleotide 101 of SEQ ID NO:32; nucleotide 201 of SEQ ID NO:33, nucleotide 1466 of SEQ ID NO:34; or nucleotide 75 of SEQ ID NO:35, or a nucleotide sequence complementary thereto. Such combinations of oligonucleotides as disclosed herein provide a tool useful for practicing the methods of the invention such that an inference can be made as to whether a subject is likely to respond or not respond to paclitaxel therapy. Accordingly, the present invention provides kits containing such pluralities of oligonucleotides, including kits containing oligonucleotides useful as hybridization probes; kits containing oligonucleotides useful, alone, as a primer for a primer extension reaction, or in combination as amplification primer pairs for an amplification reaction; or combinations of such probes and primers.

The present invention also relates to a plurality of nucleic acid molecules at least two nucleic acid molecules, each containing a SNP, the nucleotide occurrence of which allows an inference as to whether a subject is likely to respond, or not respond, to paclitaxel. The plurality of nucleic acid molecule includes, for example, a) at least one nucleotide sequence of a cytochrome P450 (CYP) gene comprising such a SNP, wherein the nucleotide sequence includes nucleotides of a CYP2C8 gene, including a nucleotide corresponding to nucleotide 83 of SEQ ID NO:1, nucleotide 251 of SEQ ID NO:2; nucleotide 181 of SEQ ID NO:15, or nucleotide 75 of SEQ ID NO:35; nucleotides of a CYP3A4 gene, including a nucleotide corresponding to nucleotide 401 of SEQ ID NO:3, nucleotide 437 of SEQ ID NO:4; nucleotide 151 of SEQ ID NO:16, or nucleotide 1466 of SEQ ID NO:34; nucleotides of a CYP3A7 gene, including a nucleotide corresponding to nucleotide 401 of SEQ ID NO:9, nucleotide 541 of SEQ ID NO:10, nucleotide 151 of SEQ ID NO:17, or nucleotide 201 of SEQ ID NO:21; nucleotides of a CYP3A5 gene, including a nucleotide corresponding to nucleotide 201 of SEQ ID NO:13, or nucleotide 101 of SEQ ID NO:14; nucleotides of a CYP2D6 gene, including a nucleotide corresponding to nucleotide 201 of SEQ ID NO:23; nucleotides of a CYP4B gene, including a nucleotide corresponding to nucleotide 123 of SEQ ID NO:29; nucleotides of a CYP2C9 gene, including a nucleotide corresponding to nucleotide 122 of SEQ ID NO:19, or a nucleotide sequence complementary to any of the foregoing, and b) at least one nucleotide sequence of a gene (other than a CYP gene) containing a SNP, the gene having a nucleotide sequence including nucleotides of an ESD gene, including a nucleotide corresponding to nucleotide 702 of SEQ ID NO:5, or nucleotide 201 of SEQ ID NO:6; nucleotides of a GSTM1 gene, including a nucleotide corresponding to nucleotide 201 of SEQ ID NO:7, or nucleotide 191 of SEQ ID NO:8; nucleotides of an MAOB gene, including a nucleotide corresponding to nucleotide 501 of SEQ ID NO:11, or nucleotide 60 of SEQ ID NO:12; nucleotides of an ASIP gene, including a nucleotide corresponding to nucleotide 201 of SEQ ID NO:22, nucleotide 26 of SEQ ID NO:30, or nucleotide 101 of SEQ ID NO:32; nucleotides of a TUBB gene, including a nucleotide corresponding to nucleotide 61 of SEQ ID NO:18; nucleotides of a TYRP gene, including a nucleotide corresponding to nucleotide 592 of SEQ ID NO:20; nucleotides of an AIM gene, including nucleotide 201 of SEQ ID NO:24; nucleotides of a GSTT gene, including a nucleotide corresponding to nucleotide 61 of SEQ ID NO:25; nucleotides of a DCT gene, including a nucleotide corresponding to nucleotide 201 of SEQ ID NO:26, or nucleotide 61 of SEQ ID NO:27; nucleotides of an OCA gene, including a nucleotide corresponding to nucleotide 135 of SEQ ID NO:28; nucleotides of a POR gene, including a nucleotide corresponding to nucleotide 61 of SEQ ID NO:31; nucleotides of an RAB gene, including a nucleotide corresponding to nucleotide 201 of SEQ ID NO:33, or a nucleotide sequence complementary to any of the foregoing.

In one aspect of an embodiment of the present invention, the plurality of nucleic acid molecules includes nucleotides of a CYP gene, for example, nucleotides of a CYP2C8 gene, including a nucleotide corresponding to nucleotide 83 of SEQ ID NO:1, nucleotide 251 of SEQ ID NO:2; nucleotide 181 of SEQ ID NO:15, or nucleotide 75 of SEQ ID NO:35; nucleotides of a CYP3A4 gene, including a nucleotide corresponding to nucleotide 401 of SEQ ID NO:3, nucleotide 437 of SEQ ID NO:4; nucleotide 151 of SEQ ID NO:16, or nucleotide 1466 of SEQ ID NO:34; nucleotides of a CYP3A7 gene, including a nucleotide corresponding to nucleotide 401 of SEQ ID NO:9, nucleotide 541 of SEQ ID NO:10, nucleotide 151 of SEQ ID NO:17, or nucleotide 201 of SEQ ID NO:21; nucleotides of a CYP3A5 gene, including a nucleotide corresponding to nucleotide 201 of SEQ ID NO:13, or a nucleotide sequence complementary to any of the foregoing. In another aspect of the embodiment, the plurality of nucleic acid molecules includes nucleotides of an ESD gene, including a nucleotide corresponding to nucleotide 702 of SEQ ID NO:5, or nucleotide 201 of SEQ ID NO:6; nucleotides of a GSTM1 gene, including a nucleotide corresponding to nucleotide 201 of SEQ ID NO:7, or nucleotide 191 of SEQ ID NO:8; nucleotides of a MAOB gene, including a nucleotide corresponding to nucleotide 501 of SEQ ID NO:11, or nucleotide 60 of SEQ ID NO:12.

In one aspect of another embodiment, the plurality of nucleic acid molecules includes nucleotide 83 of SEQ ID NO:1, nucleotide 251 of SEQ ID NO:2, nucleotide 401 of SEQ ID NO:3, nucleotide 702 of SEQ ID NO:5, nucleotide 201 of SEQ ID NO:6, nucleotide 201 of SEQ ID NO:7, nucleotide 191 of SEQ ID NO:8, nucleotide 401 of SEQ ID NO:9, nucleotide 541 of SEQ ID NO:10, nucleotide 501 of SEQ ID NO:11, nucleotide 60 of SEQ ID NO:12, nucleotide 201 of SEQ ID NO:13, nucleotide 101 of SEQ ID NO:14, nucleotide 181 of SEQ ID NO:15, nucleotide 151 of SEQ ID NO:16, nucleotide 151 of SEQ ID NO:17, and nucleotide 75 of SEQ ID NO:35; or a nucleotide sequence complementary to any of the foregoing. In another aspect of this embodiment, the plurality of nucleic acid molecules includes nucleotide 83 of SEQ ID NO:1, nucleotide 251 of SEQ ID NO:2, nucleotide 401 of SEQ ID NO:3, nucleotide 437 of SEQ ID NO:4, nucleotide 702 of SEQ ID NO:5, nucleotide 201 of SEQ ID NO:6, nucleotide 201 of SEQ ID NO:7, nucleotide 191 of SEQ ID NO:8, nucleotide 401 of SEQ ID NO:9, nucleotide 541 of SEQ ID NO:10, nucleotide 501 of SEQ ID NO:11, nucleotide 60 of SEQ ID NO:12, nucleotide 201 of SEQ ID NO:13, nucleotide 101 of SEQ ID NO:14, nucleotide 181 of SEQ ID NO:15, nucleotide 151 of SEQ ID NO:16, nucleotide 151 of SEQ ID NO:17, nucleotide 61 of SEQ ID NO:18, nucleotide 122 of SEQ ID NO:19, nucleotide 26 of SEQ ID NO:30, and nucleotide 75 of SEQ ID NO:35, or a nucleotide sequence complementary to any of the foregoing.

In still another embodiment, a nucleotide sequence of a nucleic acid molecule of the plurality, when present, includes a particular nucleotide occurrence, for example, wherein nucleotide 83 of SEQ ID NO:1 is A or G; nucleotide 251 of SEQ ID NO:2 is G or A; nucleotide 401 of SEQ ID NO:3 is C or T; nucleotide 437 of SEQ ID NO:4 is T or G; nucleotide 702 of SEQ ID NO:5 is C or T; nucleotide 201 of SEQ ID NO:6 is C or G; nucleotide 201 of SEQ ID NO:7 is C or T; nucleotide 191 of SEQ ID NO:8 is C or T; nucleotide 401 of SEQ ID NO:9 is C or T; nucleotide 541 of SEQ ID NO:10 is G or C; nucleotide 501 of SEQ ID NO:11 is C or T; nucleotide 60 of SEQ ID NO:12 is C or T; nucleotide 201 of SEQ ID NO:13 is A or G; nucleotide 101 of SEQ ID NO:14 is C or T; nucleotide 181 of SEQ ID NO:15 is A or G; nucleotide 151 of SEQ ID NO:16 is C or T; nucleotide 151 of SEQ ID NO:17 is C or T; nucleotide 61 of SEQ ID NO:18 is A or G; nucleotide 122 of SEQ ID NO:19 is A or T; nucleotide 26 of SEQ ID NO:30 is A or G; and nucleotide 75 of SEQ ID NO:35 is A or G, or a nucleotide sequence complementary to any of the foregoing.

The plurality of nucleic acid molecules can be maintained separately or in a mixture of two or more nucleic acid molecules. In addition, each nucleic acid molecule of the plurality is attached to a solid support, for example, glass slide or microchip, wherein the nucleic acid molecules can be organized in an array, which can be an addressable array. Accordingly, the invention also provides a kit, which contains a plurality of nucleic acid molecules as described above, including, for example, a kit containing a solid support to which the plurality of nucleic acid molecules is attached. A kit of the invention also can contain, for example, one or more oligonucleotides that selectively hybridizes at or near the single nucleotide polymorphism of a nucleic acid molecule of the plurality.

The present invention also relates to a plurality of oligonucleotides, which includes a) at least one oligonucleotide that selectively hybridizes at or near a SNP of a nucleotide sequence of a CYP gene, for example, nucleotides of a CYP2C8 gene, including a nucleotide corresponding to nucleotide 83 of SEQ ID NO:1, nucleotide 251 of SEQ ID NO:2; nucleotide 181 of SEQ ID NO:15, or nucleotide 75 of SEQ ID NO:35; nucleotides of a CYP3A4 gene, including a nucleotide corresponding to nucleotide 401 of SEQ ID NO:3, nucleotide 437 of SEQ ID NO:4; nucleotide 151 of SEQ ID NO:16, or nucleotide 1466 of SEQ ID NO:34; nucleotides of a CYP3A7 gene, including a nucleotide corresponding to nucleotide 401 of SEQ ID NO:9, nucleotide 541 of SEQ ID NO:10, nucleotide 151 of SEQ ID NO:17, nucleotide 201 of SEQ ID NO:21; or nucleotides of a CYP3A5 gene, including a nucleotide corresponding to nucleotide 201 of SEQ ID NO:13, or nucleotide 101 of SEQ ID NO:14; nucleotides of a CYP2D6 gene, including a nucleotide corresponding to nucleotide 201 of SEQ ID NO:23; nucleotides of a CYP4B gene, including a nucleotide corresponding to nucleotide 123 of SEQ ID NO:29; or nucleotides of a CYP2C9 gene, including a nucleotide corresponding to nucleotide 122 of SEQ ID NO:19, or a nucleotide sequence complementary to any of the foregoing, and b) at least one oligonucleotide that selectively hybridizes at or near a SNP of a nucleotide sequence of a gene containing the SNP, for example, to nucleotides of an ESD gene, including a nucleotide corresponding to nucleotide 702 of SEQ ID NO:5, or nucleotide 201 of SEQ ID NO:6; nucleotides of a GSTM1 gene, including a nucleotide corresponding to nucleotide 201 of SEQ ID NO:7, or nucleotide 191 of SEQ ID NO:8; nucleotides of an MAOB gene, including a nucleotide corresponding to nucleotide 501 of SEQ ID NO:11, or nucleotide 60 of SEQ ID NO:12; nucleotides of an ASIP gene, including a nucleotide corresponding to nucleotide 201 of SEQ ID NO:22, nucleotide 26 of SEQ ID NO:30, or nucleotide 101 of SEQ ID NO:32; nucleotides of a TUBB gene, including a nucleotide corresponding to nucleotide 61 of SEQ ID NO:18; nucleotides of a TYRP gene, including a nucleotide corresponding to nucleotide 92 of SEQ ID NO:20; nucleotides of an AIM gene, including nucleotide 201 of SEQ ID NO:24; nucleotides of a GSTT gene, including a nucleotide corresponding to nucleotide 61 of SEQ ID NO:25; nucleotides of a DCT gene, including a nucleotide corresponding to nucleotide 201 of SEQ ID NO:26, or nucleotide 61 of SEQ ID NO:27; nucleotides of an OCA gene, including a nucleotide corresponding to nucleotide 135 of SEQ ID NO:28; nucleotides of a POR gene, including a nucleotide corresponding to nucleotide 61 of SEQ ID NO:31; or nucleotides of an RAB gene, including a nucleotide corresponding to nucleotide 201 of SEQ ID NO:33, or a nucleotide sequence complementary to any of the foregoing.

The oligonucleotides of the plurality can be oligonucleotide probes, which selectively hybridize to a nucleotide sequence including the position of the SNP, or can be an oligonucleotide comprises a primer, which selectively hybridizes to a nucleotide sequence containing the SNP and can be extended by a polymerase in the direction of the SNP position. In one embodiment, primers of the plurality include a primer of an amplification primer pair, wherein the amplification primer pair include a forward primer and reverse primer that allow amplification of a nucleic acid molecule, including the nucleotide occurrence of a SNP in a nucleotide sequence to which the primer pair can selectively hybridize. Accordingly, the present invention also provides a plurality of such oligonucleotides, wherein each oligonucleotide of the plurality is attached to a solid support, for example, a glass slide or microchip. In one embodiment, the oligonucleotides of the plurality are attached to the solid support in an array, and in one aspect of this embodiment, the array is an addressable array. The invention further provides a kit containing a plurality of oligonucleotides as set forth above, for example, a plurality of oligonucleotides, wherein some or all of the oligonucleotides are attached to a solid support.

### DETAILED DESCRIPTION OF THE INVENTION

Paclitaxel (Taxol®) is a chemotherapeutic agent that is used to treat various cancers, including ovarian cancer and breast cancer. Paclitaxel is metabolized in the human body, in this case by the cytochrome P450 family member, CYP2C8 and, to a lesser extent, CYP3A4 (Shou et al., Eur. J. Pharmacol. 394:199-209, 2000; Dai et al., Pharmacogenetics 11:597-607, 2001, each of which is incorporated herein by reference). Paclitaxel generally is used in combination with carboplatin (paclitaxel/carboplatin; PC) as a first line treatment for treating ovarian cancer patients, with about two-thirds of such patients showing a positive response to PC treatment. Although the molecular basis for non-responsiveness to PC has not been clearly defined, variability in the sequences of enzymes involved in drug metabolism is known to be associated with variability in drug responsiveness. As disclosed herein, an examination of SNPs within common xenobiotic metabolism genes revealed a link to PC responsiveness of patients (Examples 1 and 2).

Accordingly, the present invention provides genetic markers, including SNPs and combinations of SNPs, that are predictive of PC response (and non-response), and further provides methods to individualize treatment protocols for a cancer patient by determining whether the patient is likely to respond to PC therapy. The compositions of the invention include combinations of nucleic acid molecules, each of which contains a SNP that allows an inference as to paclitaxel responsiveness, and combinations of oligonucleotides that are useful as probes or primers for detecting and/or determining the nucleotide occurrence of such a SNP. The term “nucleotide occurrence” is used herein to refer to the particular nucleotide (i.e., A, C, G, or T) at a SNP position. The methods of the invention, which include determining a nucleotide occurrence of a SNP associated with paclitaxel responsiveness, allow an inference as to whether a subject, for example, a cancer patient, is likely to respond to paclitaxel therapy or is likely not to respond.

The genetic sequences for the disclosed marker sets permit the synthesis of probes that can be used as the basis for a predictive test, including, for example, as a component of a diagnostics kit. Such probes can comprise oligonucleotide sequences that hybridize to a nucleotide sequence including the SNP position, or can comprise primer pairs that flank the SNP position. Such oligonucleotide probes and primers, which can be designed based, for example, on SEQ ID NOS:1 to 35 disclosed herein, selectively hybridize to the target nucleotide sequence under stringent conditions, which preclude substantial cross-hybridization to unrelated gene sequences. Methods for selecting such probes or primers are well known in the art, and can be determined empirically or using mathematical formulas (see, for example, Sambrook et al., “Molecular Cloning: A laboratory manual” (Cold Spring Harbor Laboratory Press 1989), which is incorporated herein by reference).

Mutations and SNPs in the CYP3A gene have been shown to contribute to a variable response to some drugs. As disclosed herein, SNPs have now been associated with predictiveness to paclitaxel responsiveness. Accordingly, the present invention provides nucleotide markers and marker sets that are predictive of a cancer patients responsiveness to treatment with paclitaxel, methods of using such markers and marker sets as an aid to determine a treatment protocol for a cancer patient, and kits containing at least one probe useful for detecting the presence or absence of a marker associated with predictiveness of paclitaxel responsiveness of a cancer patient, including kits containing a set of probes useful for detecting a maker set predictive of paclitaxel responsiveness. It will be recognized that the methods disclosed herein can be used for identifying markers and marker sets predictive of a cancer patient's responsiveness to a cancer therapeutic agent.

The reported response rate for combination paclitaxel and carboplatin (PC) therapy is about 62%, and it is generally accepted that refraction to first line chemotherapy is linked to reduced time-to-relapse and reduced long-term survival. Since an explanation for the response rate to PC therapy can be due to the wide variation in paclitaxel clearance rates observed among patients, combined with a narrow therapeutic index, xenobiotic metabolism and paclitaxel target genes were screened in an effort to identify SNPs and/or haplotypes linked with PC response. As disclosed herein, genetic features of variable response were identified, thus allowing a classification of PC response proclivity prior to initiating first line chemotherapy. Specifically, genetic markers have been identified that provide a tool for identifying potential non-responders prior to treatment, thus allowing a determination to be made of offering an alternative treatment instead. Such an “individualized” or “personalized” approach to treatment can provide significant benefit to a patient

As disclosed herein, the sequences of at least seven genes are associated with and predictive for variable PC response (see Table 6). Furthermore, when considered within the context of a complex (multifactorial) framework, about 98% of the variability in patient response is explained. These results are not likely a reflection of genetic structure within the study sample because most of the SNPs and genes that were examined were not associated with variable PC response. Instead, specific haplotypes and/or multilocus genotypes for seven of the genes examined clearly were linked with the outcome of PC treatment. Although it was difficult to make classifications using the identified sequences without their distribution statistics, because most patients harbored response sequences for some of the seven genes but non-response sequences for others, the classification was straightforward when using a linear discriminate method to represent individuals and responder groups as complex vectors and to classify individual patients based on their Euclidian distance from the mean of both groups (see Example 2).

Individual SNPs and, even more so, haplotypes of the seven genes (Table 6) allowed an inference as to paclitaxel response. Remarkably, however, the use of multilocus genotypes as a classifier categorized all of the responders correctly and all but one of the non-responders correctly (Table 8). As such, this classifier is particularly useful because it identifies most of the non-responders without incorrectly categorizing responders. Potential non-responders are those ovarian cancer patients whom can benefit from alternative therapy or adjusted dose. Because all of the responders are correctly classified, the benefit of providing alternative treatments to the potential non-responders can be realized without misdirecting individuals who would have responded to the standard regimen.

About one-third of ovarian cancer patients fail to respond to PC chemotherapy. By using the disclosed classifier before treatment, most non-responders can be identified (without misclassifying responders as non-responders), thus allowing alternative treatment of the potential non-responders. If the non-responders are eliminated from first line PC treatment, the PC response rate would become virtually 100%. Furthermore, if the classified non-responders are guided towards alternative therapies or altered dosages that have a success rate similar to that of PC treatment of the general population (e.g., 70%), the classifier would improve the overall first line success rate from 63% to about 89%. Thus, the present invention provides a classifier that can be used to personalize first line chemotherapy for an enhancement of the efficacy of combination PC chemotherapy. These results indicate that similar tests can be developed for other chemotherapy drugs such that the best drugs can be selected individually for each cancer patient. The set of markers as disclosed herein constitute a haplotype system, which is the set of diploid, phase-known haplotype combinations present in the human population. A gene containing 4 SNPs has a large number of 2-locus haplotype systems, a smaller number of 3-locus haplotype systems and one 4 locus haplotype system. Haplotype systems are comprised of polymorphic markers, which are specific sequences at a particular position in genomic DNA where individuals differ from one another (i.e., a polymorphism).

The present invention provides four newly described SNPs, including SEQ ID NO:1, wherein nucleotide 83 is G; SEQ ID NO:3, wherein nucleotide 401 is T; SEQ ID NO:4, wherein nucleotide 437 is G; and SEQ ID NO:8, wherein nucleotide 191 is T; and further provides combinations of gene nucleotide sequences, each of which includes a SNP associated with paclitaxel responsiveness. Also provided are oligonucleotides useful for determining the nucleotide occurrence at a desired SNP position, including oligonucleotide probes and primers. The term “SNP” or “SNP position” is used herein in its conventional sense to mean a position in a nucleotide sequence of a gene that exists in two or more variant forms in a population. Thus, a SNP can be, for example, A or G; or C or T; or A, G or T; etc. As such, it will be recognized that reference herein to a SNP comprising, for example, nucleotide 83 of SEQ ID NO:1, means a nucleotide sequence as set forth in SEQ ID NO:1, including at least that portion containing nucleotide 83, which, as disclosed herein, can be A or G. Further, the SNP can be described with respect to a particular nucleotide occurrence, for example, wherein nucleotide 83 of SEQ ID NO:1 is G. As such, each of the polynucleotides as set forth in SEQ ID NOS:1 to 35 corresponds to two nucleic acid molecules, each of which is polymorphic at a specified position.

As used herein, the term “associated with paclitaxel responsiveness”, when used in reference to a SNP (as well as to a haplotype allele or to diploid haplotype alleles), means that one or more nucleotide occurrences at the SNP position (or haplotype allele, or diploid haplotype alleles) allows an inference to be drawn that a subject is likely to respond to paclitaxel treatment or is likely not to respond to paclitaxel treatment. For example, that SNP at nucleotide 83 of SEQ ID NO:1 is associated with paclitaxel responsiveness and, more specifically, the occurrence of an A at nucleotide 83 of SEQ ID NO:1 allows an inference to be drawn that a subject having the nucleotide occurrence is likely to respond to paclitaxel treatment. Further, the occurrence of a G at nucleotide 83 of SEQ ID NO:1 allows an inference to be drawn that a subject is a non-responder with respect to paclitaxel.

As used herein, the term “haplotype” refers to a grouping of two or more SNPs in a single gene. The term “haplotype alleles” is used herein to refer to a non-random combination of nucleotide occurrences of SNPs that make up a haplotype. For example, SEQ ID NOS:1 and 2 are two nucleotide sequences present in the human CYP2C8 gene, each of which contains a SNP (nucleotide 83 of SEQ ID NO:1 and nucleotide 251 of SEQ ID NO:2). The SNP at nucleotide 83 of SEQ ID NO:1 can be A or G, and the SNP at nucleotide 251 of SEQ ID NO:2 also can be A or G. As such, a haplotype of the CYP2C8 gene comprising these SNPs can be represented, for example, by AA, or AG, or GG, or GA; however, it should be noted that, while the specified bases are present within the same gene, they are not contiguous in the gene. As disclosed herein, the identification of a CYP2C8 gene haplotype allele other than GA or GG, allows an inference to be drawn that the subject is a paclitaxel responder.

The term “diploid haplotype alleles” or “multilocus genotype” is used herein to refer to both alleles of a haplotype, i.e., the haplotype present on both chromosomes. A diploid haplotype allele of the CYP2C8 gene, for example, can be represented by GG/NN, where one allele contains a G at the position corresponding to nucleotide 83 of SEQ ID NO:1 and a G at the position corresponding to nucleotide 251 of SEQ ID NO:2, and wherein the other allele contains any nucleotide (N) at these positions (see Table 7). The term “corresponding” is used herein, in reference to a nucleotide position of any of SEQ ID NOS:1 to 35, to refer to the endogenous nucleotide position present in a genome, particularly a human genome.

The disclosed combinations of gene nucleotide sequences, including the SNP position, and/or oligonucleotides provide tools for screening a cancer patient, prior to initiating paclitaxel therapy, in order to determine whether the patient is likely to respond to such treatment. Where a determination is made that the patient is likely to be a paclitaxel non-responder, the clinician is in a position to consider whether the patient can benefit from an alternative therapy. The combinations of gene nucleotide sequences and oligonucleotides will be useful to medical diagnostic laboratories, and provided as separate or mixed reagents suitable for individual or high throughput assays.

An oligonucleotide probe or primer is selected such that is can selectively hybridize at or near a SNP that allows an inference as to paclitaxel responsiveness. The term “target nucleic acid molecule” is used generally herein to refer to nucleotide sequence that includes a SNP that allows an inference as to paclitaxel responsiveness. A target nucleic acid molecule can be single stranded or double stranded, and can be DNA, RNA, or a DNA/RNA hybrid. SEQ ID NOS:1 to 35, each of which comprises a SNP that, alone or in combination, allows an inference as to paclitaxel responsiveness, are provided as examples of target nucleic acid molecules.

Based on the disclosed gene sequences set forth as SEQ ID NOS:1 to 35, including the positions of the relevant SNPs and nucleotide occurrences associated with paclitaxel responsiveness, oligonucleotide probes that can selectively hybridize to a nucleotide sequence comprising the SNP (or a nucleotide sequence complementary thereto), and primers, including amplification primer pairs that flank a SNP, readily can be prepared. As used herein, the term “selective hybridization” or “selectively hybridize” refers to hybridization under moderately stringent or highly stringent conditions such that a nucleotide sequence associates with a complementary sequence of a selected target nucleic acid molecule, but not with unrelated nucleotide sequences. Generally, an oligonucleotide useful as a probe or primer that selectively hybridizes to a selected nucleotide sequence is at least about 15 nucleotides in length, usually at least about 18 nucleotides, and particularly about 21, 22, 23, 24, 25, or 30 nucleotides in length or more in length. Conditions that allow for selective hybridization can be determined empirically, for example, by determining specificity of an oligonucleotide probe for a target nucleic acid molecule with respect to a nucleotide sequence of a gene related to the target nucleic acid molecule (e.g., by determining that the probe or primer is specific for a target cytochrome P450 nucleotide sequence, but not for nucleotide sequence of other related cytochrome P450 gene family members). Conditions that allow for selective hybridization also can be estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual (Cold Spring Harbor Laboratory Press 1989), which is incorporated herein by reference).

The presence of a particular SNP can be detected using a hybridization method wherein an oligonucleotide probe hybridizes, for example, to a target nucleic acid molecule containing a particular nucleotide occurrence of a SNP, but not other nucleotide occurrences (e.g., A, but not C, G, or T). Selective hybridization of the probe can be detected, for example, by gel electrophoresis, wherein the probe or a cleave product thereof is identified. For example, where the probe hybridizes to a nucleotide sequence comprising the SNP position, but contains a mismatch with respect to the SNP, contact with an endonuclease can cleave the probe, the cleavage products of which readily can be identified by having a faster migration in a gel than the full length oligonucleotide probe. Conversely, an oligonucleotide ligation assay also can be used to identify a particular nucleotide at a SNP position. Such reaction can utilize an oligonucleotide probe comprising two portions, including a first portion that hybridizes 5′ and up to and including the position of the SNP and a second portion that hybridizes immediately 3′ to the SNP. When the nucleotide of the probe portion in the position complementary to the SNP is complementary to the nucleotide actually at the SNP position, and the hybridizing sequences are further contacted with a DNA ligase, the first and second probe portions are ligated to generate a product that migrates more slowly in a gel than the separate first and second probe portions.

An oligonucleotide probe also can be a bilabeled oligonucleotide probe such as a molecular beacon or a TaqMan™ probe, which includes a fluorescent moiety and a fluorescence quencher moiety, wherein the bilabeled oligonucleotide probe can selectively hybridize to a nucleotide sequence of the target nucleic acid molecule comprising the SNP position; and detecting fluorescence due to the fluorescent moiety. Such a method, when combined with an amplification reaction (see below), provides a means for real-time detection of the generation of the amplification product.

The presence of a particular SNP also can be detected using a primer extension reaction or amplification reaction. For example, a nucleic acid sample containing (or suspected of containing) a target nucleic acid molecule can be contacted with an oligonucleotide primer that, upon further contact with a polymerase, can be extended up to and, if desired, beyond the position of the SNP. In addition, the nucleic acid sample can be contacted with an amplification primer pair, comprising a first primer and a second primer, which selectively hybridize to complementary strands of a target nucleic acid molecule and, in the presence of polymerase, allow for generation of an amplification product. For convenience, the primers of an amplification primer pair are referred to as a “first primer” and a “second primer”; however, reference herein to a “first primer” or a “second primer” is not intended to indicate any importance, order of addition, or the like. It will be further recognized that an amplification primer pair requires that the first and second primer comprise what are commonly referred to as a forward primer and a reverse primer, which, in view, for example, of SEQ ID NOS:1 to 35, can be selected using well known and routine methods such that an amplification product can be generated.

A primer extension or PCR amplification reaction can be designed such that the presence of a particular nucleotide at a SNP position can be determined by the presence or size of the extension and/or amplification product, in which case the SNP can be determined using a method such as gel electrophoresis, capillary gel electrophoresis, or mass spectrometry; or the amplification product can be sequenced to determine the nucleotide at the SNP position. In addition, the SNP can be detected indirectly, for example, by further contacting the sample with a detector oligonucleotide, which can selectively hybridize to a nucleotide sequence of the first amplification product comprising the SNP position; and detecting selective hybridization of the detector oligonucleotide, as above.

Various endpoint detection formats are known to the art and can be applied to the present methods. For example, PCR can be performed using TaqMan™ reagents, followed by reading the plates at this endpoint. Molecular beacons, Amplifluor™ or TriStar™ reagents and methods similarly can be used (Stratagene; Intergen). Amplification products also can be detected using an ELISA format, for example, using a design in which one primer is biotinylated and the other contains digoxygenin. The amplification products are then bound to a streptavidin plate, washed, reacted with an enzyme-conjugated antibody to digoxygenin, and developed with a chromogenic, fluorogenic, or chemiluminescent substrate for the enzyme. Alternatively, a radioactive method can be used to detect generated amplification products, for example, by including a radiolabeled deoxynucleoside triphosphate into the amplification reaction, then blotting the amplification products onto DEAE paper for detection. In addition, if one primer is biotinylated, then streptavidin-coated scintillation proximity assay plates can be used to measure the PCR products. Additional methods of detection can use a chemiluminescent label, for example, a lanthanide chelate such as used in the DELFIA® assay (Pall Corp.), an electrochemiluminescent label such as ruthenium tris-bipyridy (ORI-GEN), or a fluorescent label, for example, using fluorescence correlation spectroscopy.

An assay system that is commercially available and can be used to identify a nucleotide occurrence of one or more SNPs is the SNP-IT™ assay system (Orchid BioSciences, Inc.; Princeton N.J.). In general, the SNP-IT™ method is a three step primer extension reaction. In the first step a target nucleic acid molecule is isolated from a sample by hybridization to a capture primer, which provides a first level of specificity. In a second step the capture primer is extended from a terminating nucleotide triphosphate at the target SNP site, which provides a second level of specificity. In a third step, the extended nucleotide triphosphate can be detected using a variety of known formats, including, for example, by direct fluorescence, indirect fluorescence, an indirect colorimetric assay, mass spectrometry, or fluorescence polarization. Reactions conveniently can be processed in 384 well format in an automated format using a SNPstream™ instrument (Orchid BioSciences, Inc.).

The methods of the invention are readily adaptable to high throughput assays. For example, an amplification reaction such as PCR can be performed using inexpensive robotic thermocyclers for a specified number of cycles, then the amplification product generated can be determined at the endpoint of the reaction. Furthermore, the methods can be performed in a multiplex format, for example, using differentially labeled oligonucleotide probes, or performing oligonucleotide ligation assays that result in different sized ligation products, or amplification reactions that result in different sized amplification products.

The oligonucleotide probes and/or primers, and gene nucleotide sequences comprising one or all nucleotide occurrences of a SNP associated with paclitaxel responsiveness (e.g., SEQ ID NOS:1 to 35) can be provided as isolated nucleic acid molecules, for example, in a kit, or can be attached to a solid support such as a microchip, a glass slide, a membrane, or a bead. Methods for immobilizing nucleotide sequences on a solid support, particularly a support such as a microchip or glass slide, are well known (see, for example, DeRisi et al., Science 278:680-686, 1997; Marton et al., Nat. Med. 4:1293-1301, 1998; Lipshutz et al., Nat. Genet. 21:20-24, 1999, each of which is incorporated herein by reference). Preferably, the nucleotide sequences are immobilized in an addressable array, wherein each nucleotide sequence (either gene sequence including a SNP, or oligonucleotide probe or primer) is present in a defined position in the array, thus facilitating identification of particular nucleotide occurrences of SNPs in a test nucleic acid sample.

The compositions of the invention conveniently can be provides as a kit, which can contain, for example, two or more oligonucleotides useful for detecting nucleotide occurrences at the SNP positions of two or more genes, wherein each SNP provides an inference as to paclitaxel responsiveness. The oligonucleotides of the kit can be oligonucleotide probes comprising a nucleotide sequence of at least two of SEQ ID NOS:1 to 35 comprising the SNP position, particularly oligonucleotide probes specific for at least one cytochrome gene and one gene other than a cytochrome gene. The term “at least one” means one or more. As such, the term includes 1, 2, 3, 4, 5, 6, 7, 8, 9, 10, etc. and, can include, for example, all of the exemplified SNPs, haplotype alleles, diploid haplotype alleles, and oligonucleotide probes and/or primers. Similarly, the term “at least two” means two or more, e.g., 2, 3, 4, 5, etc.

The oligonucleotides of the kit also can be oligonucleotide primers (one or all of which can be first amplification primers of an amplification primer pair) that can be used to generate extension (and/or amplification) products of at least two different target nucleic acid molecules, including the nucleotides present at the SNP positions; or can be combinations of such probes and primers (and primer pairs). It should be recognized that each of the nucleotide sequences set forth as SEQ ID NOS:1 to 35, or nucleotide sequences complementary thereto, can be useful, in whole or as a portion containing the SNP position, as probes for detecting particular nucleotide occurrences of SNPs associated with paclitaxel responsiveness in a nucleic acid sample.

A kit of the invention also can contain one or more reagents useful for practicing a method of the invention. For example, the kit can contain a control target nucleic acid molecule for a probe or primer contained in the kit. A kit of the invention also can contain one or more detectable labels that can be used, alone or in combination, with the probes and/or primers, wherein the kit can further contain reagents for linking the label to the probe or primer. A kit containing a variety of detectable labels can be useful, for example, for preparing probes for a multiplex analysis assay. Alternatively, the probes of a kit can be provided as labeled probes, e.g., fluorescently labeled probes, and can further be bilabeled oligonucleotide probes containing a fluorescent moiety and a quenching moiety, which, when in proximity to the fluorescent moiety, quenches fluorescence.

The following examples are intended to illustrate the present invention.

### EXAMPLE 1

### Identification of SNPs Predictive of Paclitaxel Response

This example provides a method of identifying a marker set of SNPs that are predictive of paclitaxel responsiveness of a cancer patient.

A large number of haplotype systems within 30 xenobiotic metabolism genes were screened. Within the CYP3A4 gene in particular, 50 haplotype systems were randomly selected and tested for association with paclitaxel response (see, for example, Table 1, showing SNPs of CYP3A4 gene screened). Of these 50 haplotype systems, one system, TX3A41119 (CYP3A4 gene) was identified whose constituent haplotypes were linked with paclitaxel response (Tables 1 and 2; see Sequence Listing for nucleotide sequences flanking the SNPs).

TABLE 1

| SNPNAME | MARKER | LOCATION | GENBANK | INTEGRITY | TYPE |
|---|---|---|---|---|---|
| CYP3A4E3-5_249 | 809114 | 6165 | AF209389 | POLY | INTRON |
| [symbol] | [symbol] | [symbol] | [symbol] | [symbol] | [symbol] |
| [symbol] | [symbol] | [symbol] | [symbol] | [symbol] | [symbol] |
| CYP3A4E8E9-5_460 | 809121 | 17303 | AF209389 | POLY | INTRON |
| CYP3A4E10-5_292 | 712037 | 20338 | AF209389 | POLY | INTRON |
| CYP3A4E11-5_242 | 809111 | 22094 | AF209389 | POLY_RARE | SILENT |
| CYP3A4E12_9 | 869771 | 23120 | AF209389 | POLY_RARE | INTRON |
| [symbol] | [symbol] | [symbol] | [symbol] | [symbol] | [symbol] |

Table 1 shows the CYP3A4 polymorphisms tested for association with paclitaxel response in ovarian cancer patients. The name of the SNP is shown in Column 1. The unique identifier for each SNP is shown (Column 2), as is its location (Column 3) within an NCBI reference sequence (Column 4). The status of each SNP, i.e., whether it is a validated polymorphic marker (indicated by “POLY”), is shown in Column 5. Type of polymorphism, i.e., whether it is located in a coding, silent or intron region of the gene, is shown in Column 6. The haplotype system described in this Example is a combination of three SNPs shown in italics and bold print.

Ovarian Cancer patients were treated with several (up to 15) rounds of paclitaxel and carboplatin therapy. Baseline reading of the cancer associated protein, CA125, were established prior to each round, and post-treatment readings were taken within a month of each treatment in each patient. The investigation was designed to determine whether there were genetic differences in xenobiotic metabolism gene sequences between patients who responded to therapy and those who did not respond. In the first step of the study, which was designed to discover SNPs in each of the xenobiotic metabolism genes, an average of 30 SNPs in 50 such genes was identified. In the second step, the patients were genotyped at each of these SNPs. Approximately 1500 SNP positions were sequenced in 30 ovarian cancer patients to produce 450,000 genotypes.

TABLE 2

| | HAP | | | | |
|---|---|---|---|---|---|
| GENE | SYSTEM | % | LEVEL | FST P value | Exact P value |
| CYP3A4 | TX3A41119 | 20 | individual | 0.00901 +− 0.0091 | 0.08572 +− 0.00395 |
| | | 20 | test pair | 0.00901 +− 0.0091 | 0.00000 +− 0.00000 |
| | | 50 | individual | 0.03604 +− 0.0148 | 0.23224 +− 0.00285 |
| | | 50 | test pair | 0.00000 +− 0.0000 | 0.00363 +− 0.00056 |
| CYP2D6 | TX2D61120 | 20 | individual | 0.32432 +− 0.0411 | 0.55956 +− 0.00676 |
| | | 20 | test pair | 0.55856 +− 0.0504 | 0.67434 +− 0.00771 |
| | | 50 | individual | 0.60360 +− 0.0490 | 0.92720 +− 0.00206 |
| | | 50 | test pair | 0.70270 +− 0.0425 | 0.81895 +− 0.00469 |

Table 2 shows the results of differentiation tests of genetic structure between paclitaxel responders and non-responders with ovarian cancer. Analyses for haplotype systems (Column 2) within two genes (Column 1) are presented. Two criteria for response were used: a 20% and a 50% reduction in CA125 reading post-paclitaxel treatment. The analyses were performed on two levels (Column 4). The “individual level” uses an average CA125 response per individual and counts each individual only once. The “test pair” level uses each paclitaxel treatment-CA125 reading pair; any one individual may be counted several times depending on the number of treatments they received. P values for a pair-wise F-statistic (Column 4) and an Exact test of Differentiation (Column 5) are shown.

To find which marker sets within which genes were associated with paclitaxel response, a set of markers was defined, then a standard approach, which was the same for every set of markers selected, was used. The patient population was divided into two groups: 1) responders, which were defined as patients whose post treatment CA125 level was 20% or 50% lower than the baseline, and 2) non-responders. This was done on the level of individuals, where an average reading was used for each patient, and each patient was counted only once, and on the level of readings, where each patient could be counted several times. Haplotypes were inferred from the genotype combinations within each patient using the algorithm of Stephens et al. (Amer. J. Hum. Genet. 68:978-989, 2001, which is incorporated herein by reference), where the goal next was to determine whether there was a statistically significant distinction in haplotype constitution between the responder groups. If the answer was no, another set of markers was selected.

Using this process, a three-locus haplotype system (TX3A41119) was identified whose constituent elements were predictive for paclitaxel response in ovarian cancer patients (see Table 2, above). F-statistic P-values for the TX3A41119 haplotype system were highly significant, regardless of whether the 20% or 50% CA125 reduction criteria was used, and regardless of whether individual patients or readings were counted. The Exact test of differentiation, which measures genetic distinction on the level of the individual haplotype and is relatively insensitive to more complex genetic structure such as hetero/homozygosity, gave significant results when readings were counted. In contrast, no significant results were obtained for the CYP2D6 haplotype system TX2D61120, which represents a control. Several hundred haplotype systems were screened with similar results as TX2D61120.

Inspection of the haplotype pairs themselves (rather than statistical values) revealed that the CGC and CGT TX3A41119 haplotypes were associated with a non-response to paclitaxel. None of the individuals with the CGC haplotype (n=4) responded at the 50% reduction level (Table 3), and only 2 readings out of 26 from CGC containing patients were classifiable as responses; the remaining 24 were non-responses (Table 4). None of the individuals with the CGT haplotype were responders (n=3; Table 3) and all 12 readings from these 3 patients were non-responses (Table 4). In contrast, the CTC haplotype was strongly associated with paclitaxel responsiveness. Only CTC/CTC individuals were average responders (Table 3), and 35 of 37 test responses were from CTC/CTC individuals (Table 4). Since not every individual harboring the CTC/CTC genotype was a responder, other genes or physiological or environmental factors may also be involved in paclitaxel responsiveness.

TABLE 3

| SampleName=“ TX3A41119 _50 RESPONDERS” | | |
|---|---|---|
| SampleSize= 9 | | |
| SampleData={ | | |
| H1 | 9 | C T C |
| | | C T C |
| } | | |
| SampleName=“ TX3A41119 _50 NON-RESPONDERS” | | |
| SampleSize= 18 | | |
| SampleData={ | | |
| H1 | 3 | C G C |
| | | C T C |
| H2 | 12 | C T C |
| | | C T C |
| H3 | 1 | C G C |
| | | C G T |
| H4 | 2 | C T C |
| | | C G T |
| } | | |

Table 3 shows counts of TX3A41119 haplotype pairs in responders (top group) and non-responders (bottom group) considering average responses in 27 patients.

TABLE 4

SampleName=“TX3A1120_50”

| SampleSize= 37 | | |
|---|---|---|
| SampleData={ | | |
| H1 | 35 | C T C |
| | | C T C |
| H2 | 2 | C G C |
| | | C T C |
| } | | |
| SampleName=“TX3A1120_50” | | |
| SampleSize= 106 | | |
| SampleData={ | | |
| H1 | 20 | C G C |
| | | C T C |
| H2 | 74 | C T C |
| | | C T C |
| H3 | 4 | C G C |
| | | C G T |
| H4 | 8 | C T C |
| | | C G T |
| } | | |

Table 4 shows counts of TX3A41120 haplotype pairs in responders (top group) and non-responders (bottom group) considering each response in 27 patients. 143 total responses were measured.

These results demonstrate that the disclosed marker set can be used as the basis for construction of a paclitaxel patient classifier test, wherein if a patient has a CTC/CTC haplotype combination, for either the TX3A41119 or TX3A41120 haplotype system, he or she can be considered eligible for paclitaxel therapy, whereas if a patient has a CGC or CGT haplotype for either system, he or she is less likely to respond to conventional paclitaxel treatment.

### EXAMPLE 2

### Classifier for Predicting Response to Treatment With Paclitaxel and Carboplatin

This Example extends the studies described in Example 1 and identifies haplotype alleles associated with paclitaxel response or non-response.

Thirty five percent of ovarian cancer patients fail to respond to first line combination paclitaxel and carboplatin (PC) therapy. In the present study, 42 ovarian cancer patients, including 27 clinical responders and 15 non-responders, were genotyped at 746 SNPs from 38 xenobiotic metabolism genes and 3 tubulin genes (see Table 5, below). Haplotype alleles were identified in CYP2C8 (p<0.000) and CYP3A4 (p=0.005) as significantly associated with variable PC response. Haplotype alleles also were identified in GSTM1 (p=0.018), CYP3A7 (p=0.022), and ESD (p=0.017) as significantly associated with outcome, and haplotype alleles in CYP3A5 (p=0.070) and MAOB (p=0.075) were identified as being marginally associated. Linear and quadratic discriminate techniques were applied to model these genetic features for predicting PC response. Using two different clinical criteria for evaluation of response over the treatment line, the accuracy of the responder classification was 100% and the sensitivity of the non-responder classification was 93%. These results confirm and extend previous the results of Example 1 indicating that the CYP2C and CYP3A family of cytochrome oxidases are important determinants of variable PC response, and further implicate the esterase D (ESD, glutathione S transferase (GSTM1) and monoamine oxidase (MAOB) families of genes as determinants. These results further indicate that, in ovarian cancer patients, first line PC response can be a function of xenobiotic metabolism, rather than tumor type or stage, thus providing a tool for prescreening cancer patients in order to individualize and customize chemotherapy.

Study Design

A case control study design was used to measure the genetic distinction between paclitaxel/carboplatin (PC) responders and non-responders. Single nucleotide polymorphisms (SNPs) in seven xenobiotic metabolism genes were mined from the public database resource (NCBI:dbSNP, which can be accessed on the world wide web, at the URL “ncbi.nlm.nkh.gov/SNP/, which is incorporated herein by reference). In addition, deep re-sequencing was conducted by amplifying (using pfu polymerase), sequencing and computationally comparing each gene promoter, exon, and 3′ untranslated region in a racially inclusive group of 650 individuals.

Data Collection

Basic biographical and clinical data (i.e., other medications taken, tumor type, surgical stage, surgical procedures) was obtained from 42 patients with ovarian cancer who previously had undergone chemotherapy with standard doses of combination paclitaxel (175 mg/m² intravenous infusion over 3 hr) and carboplatin at a dose of AUC 5 or 6 (using the Calvert Formula), intravenous infusion over 1 hr. About one-half of the 42 patients were of self-described European ancestry (e.g., Latina), while the other half were of significant African and/or Native American ancestry. First line treatment response was assessed using circulating cancer antigen 125 (CA125) measurements. Clinical data was extracted from the patient charts and direct patient interviews with a research nurse. After reading and signing an approved IRB consent form, patients completed a biographical questionnaire and provided 4 ml of blood. DNA was extracted from circulating lymphocytes using a commercial kit (Qiagen), and amplified using nested PCR to front-end a primer extension protocol employing a 25K SNPstream™/ultra-high throughput (UHT) genotyping system (Orchid Biosciences; Princeton N.J.).

Response Measurement

Response to treatment was measured in two ways. For both methods, only treatments that were administered within the context of regular 3 week interval treatment schedules were considered. The “Average PC response” was defined as average response per cycle over the first line of treatment. To calculate each cycle response, the earliest CA125 reading after the PC cycle was subtracted from the latest CA125 reading before the PC cycle (the baseline), and the result was divided by the baseline value. Positive response was defined by at least 11% average reduction in CA125 levels over the entire line of PC treatment. An “Overall PC response” was used to measure the overall outcome for the first line treatment. In terms of Overall PC response a patient is considered a responder if their CA125 values dropped below the upper normal CA125 limit (35 units) by the end of the line of PC chemotherapy.

Statistical Analysis and Modeling

Genetic Feature Extraction

To identify useful genetic features of variable PC response, SNPs with alleles associated with response were first identified using the delta value method (see below; see, also, Chakraborty and Weiss, Proc. Natl. Acad. Sci., USA 85:9119-9123, 1988; Shriver et al., Amer. J Hum. Genet. 60:957-964, 1997, each of which is incorporated herein by reference). Individuals with missing data were eliminated from the analysis, and for feature extraction, individuals with ambiguous genotypes also were eliminated.

For more detailed analysis of phase-known SNP alleles such as that of Table 7 (see below), ambiguous SNP genotypes were estimated by eye from a 2-D genotype plot in a manner that was blind to sample response; a signal strength of at least 100 on at least one axis using the Orchid UHT data collection software was required. For each of the SNP combinations tested, phase-known allele associations, phenotype and genotype data were retrieved from an Oracle database and merged with genotype data using a proprietary java based software program. After inferring haplotype phase (Stephens et al., Amer. J Hum. Genet. 68:978-989, 2001, which is incorporated herein by reference). PC responders and non-responders were grouped and tested for whether haplotype alleles of the SNP combination was significantly associated with variable PC response.

To test the alleles of each SNP combination, F-statistic and Fishers Exact tests were used to determine whether there was a statistically significant distinction in the sequence composition between grouped responders and non-responders. To avoid having to test all possible SNP combinations within a gene for allelic association with variable PC response (a computationally intensive effort), those combinations incorporating only those SNPs selected from the delta value screen were examined. When more than one such combination was available, a list of p-values was created and the combination with the lowest value was selected. This approach identified the optimal combination of SNP loci, the phase-known alleles of which were most significantly associated with variable PC response.

Genetic Feature Modeling

Linear/Quadratic Classification

To use the haplotype alleles for the inference of PC response, a software program was written for using a parametric, multivariate linear classification and Quadratic classification technique with modifications for genomics data. Under the assumption that the samples have been taken from multivariate normal distributions with different mean vectors with common variance covariance matrix, linear classification procedures were applied (Fisher, Ann. Eugen. 7:179-188, 1936; Rao, Nature 160:835-836, 1948; Smith, Ann. Eugen. 13:272-282, 1947, each of which is incorporated herein by reference). The pooled within-population variance-covariance matrix was computed using the following equation (1):
S=Σ^{p}ᵢ₋₁Σ^{Ni}ⱼ₋₁(Yᵢⱼ−μᵢ)(Yᵢⱼ−μᵢ)′/Σ(Nᵢ−1) (1)
where Yᵢⱼ is the vector of character measurements for the j'th individual in the i'th group, and μᵢ and Nᵢ are the vector of means and sample size for the i'th group.

The components for these vectors are surrogate values for SNP, haplotype or multilocus genotype alleles, each dimension of the vector representing a different locus. For example, an individual is represented by a vector Y=(i,j, . . . n), where I and j=0 or 1 and n=the number of multilocus genotypes all of the genetic features. The generalized distance of the ij'th individual from the mean of the k'th group was computed using the following equation (2):
D²_{ij,k}=(Yᵢⱼ−μₖ)′S⁻¹(Yᵢⱼ−μₖ) for k≠i (2)
The vector Yᵢⱼ is used to calculate μₖ, the mean of it's own group. To avoid circularity caused by this method, correction when comparing an element with its own class can be used (Smouse and Neel, Genetics 85:733-752, 1977, which is incorporated herein by reference).

In the case of complex genetics, the following equation (3) was used to correct for circularity caused by comparing an individual with the mean of its own group:
D²_{ij,i}=(Nᵢ/(Nᵢ−1))² (Yᵢⱼ−μᵢ)′S⁻¹(Yᵢⱼ−μᵢ) (3)
The ij'th individual is allocated to that group for which (2)/(3) is minimum. Large “between class distances” relative to “within class differences” provide justification for using the mean vector values for each class as a classifier tool. The result of applying equations (2) and (3) is a probability matrix for the classification of individuals of each responder class into the correct and incorrect group.

To provide for the possibility that responder populations have different variance-covariance matrices (the matrices are, of course, samples of the general treatment population), a quadratic classification procedure also was implemented. The quadratic discriminant score for the i'th group was determined using the following equation (4):
D²_{ij,k}=ln |Sₖ|+(Yᵢⱼ−μₖ)′S⁻¹ₖ(Yᵢⱼ−μₖ) for k−1,2, . . . g (groups) (4)
Classification is the allocation of the ij'th individual to that group for which (4) is minimum.

Monte Carlo simulation was used to generate the distribution and summary statistics for classification probabilities of responders into the responder group, responder falling into the non-responder group, non-responders into the non-responder group, and non-responders into the responder group. Using a random number generator, 200 individuals were created on the basis of observed allele frequencies from both groups and used to calculate a multivariate linear classification probability matrix. This experiment was repeated 10,000 times to obtain the summary statistics of Classification and misclassification rates and their Confidence Intervals.

Results

Phenotyping

Forty-two ovarian cancer (OC) patients were phenotyped for first line PC response. The average patient in this study was treated with 6 cycles over the first line of PC treatment. Approximately 51% of these (21/41) had a positive Average PC response based on an average 20% decrease in CA125 from the beginning of each cycle to the end. Approximately 61% (25/41) of the patients exhibited a positive Overall PC response, based on overall CA125 reduction over the line to a value below 35. A perfect correlation was observed between Overall CA125 response and measured clinical response as determined by tumor mass reduction. The PC response rates were roughly comparable to the 63% average positive response rate previously described for PC combination therapy (see, for example, Recchi et al., Eur. J. Gynaecol. Oncol. 22:287-291, 2001; Grunland et al., Gynecol. Oncol. 83:128-134, 2000), indicating that the sample size was representative of the general patient population. Covariates such as age, weight, height, race and smoking status (active or not) were not associated with Average or Overall PC response, though smoking frequency (packs/day) was marginally associated with Average PC response. Multiple regression analysis combining these covariates, as independent variables, revealed an R-square of only 0.08. Only one of the patients was taling a drug known to be an inhibitor of a major cytochrome P450 (Verapamil —CYP3A4), but this patient was a PC responder.

Genetic Feature Extraction and Characterization

Patients were genotyped for 746 SNP sequences in 41 genes of potential relevance for paclitaxel disposition and/or action. Thirty-eight of the genes were xenobiotic metabolism genes and 3 were drug target (tubulin) genes (see Table 5, column 1). To measure the association between SNP sequence and response, the delta value, which is a measure of (minor allele:major allele) ratio difference between two populations (i.e., responders and non-responders) was used (Shriver et al., supra, 1997).

TABLE 5
Candidate SNPs tested, validated SNPs observed (frequency > 0.05), and composition of
selected feature SNP combinations predictive for Overall paclitaxel and carboplatin response in
ovarian cancer patients.

| | CANDIDATE | VALIDATED | FEATURE | | SELECTED |
|---|---|---|---|---|---|
| GENE | SNPs¹ | SNPs² | SNPs ID'd | MARKERS³ | COMBOS⁴ |
| CYP2D6 | 63 | 24 | 0 | | |
| CYP3A4 | 53 | 16 | 2 | 664803, 232 | 664803\|232 |
| CYP2C8 | 49 | 28 | 4 | 265, 357, 371, 369 | 265\|371 |
| CYP2E1 | 48 | 16 | 0 | | |
| CYP2B6 | 28 | 17 | 0 | | |
| CYP2A6 | 16 | 10 | 0 | | |
| CYP2C9 | 46 | 16 | 0 | | |
| CYP4B1 | 29 | 20 | 0 | | |
| CYP1B1 | 9 | 5 | 0 | | |
| CYP1A1 | 15 | 4 | 0 | | |
| CYP1A2 | 15 | 8 | 0 | | |
| CYP3A7 | 30 | 15 | 3 | 121, 229, 113 | 121\|113 |
| CYP3A5 | 10 | 6 | 1 | 537 | 537\|247 |
| CYP2A13 | 11 | 6 | 0 | | |
| NAT1 | 1 | 0 | 0 | | |
| NAT2 | 10 | 9 | 0 | | |
| XO | 7 | 5 | 0 | | |
| PON1 | 11 | 5 | 0 | | |
| PON2 | 1 | 0 | 0 | | |
| PON3 | 12 | 3 | 0 | | |
| POR | 10 | 6 | 0 | | |
| ABC | 14 | 5 | 0 | | |
| AHR | 21 | 13 | 0 | | |
| CES1 | 4 | 3 | 0 | | |
| CES2 | 5 | 1 | 0 | | |
| CYP2C18 | 4 | 2 | 0 | | |
| CYP2C19 | 9 | 3 | 0 | | |
| ESD | 19 | 11 | 1 | 677 | 677\|648 |
| GSTA2 | 64 | 23 | 0 | | |
| GSTM1 | 9 | 9 | 1 | 527 | 527\|200 |
| GSTM3 | 1 | 1 | 0 | | |
| GSTP1 | 10 | 6 | 0 | | |
| GSTT1 | 12 | 4 | 0 | | |
| GSTT2 | 25 | 20 | 0 | | |
| MAOA | 19 | 8 | 0 | | |
| MAOB | 22 | 4 | 1 | 465 | 465\|477 |
| UGT1A1 | 9 | 2 | 0 | | |
| UGT1A2 | 8 | 7 | 0 | | |
| TUBB1 | 10 | 2 | 0 | | |
| TUBB2 | 1 | 0 | 0 | | |
| TUBB4 | 6 | 3 | 0 | | |
| TOTAL | 746 | 346 | 13 | 13 | 14 |

¹The number of single nucleotide polymorphisms we tested for association with Overall PC response. Each was derived from private re-sequencing and/or the NCBI public database resources as described in the text.
²The number of validated SNPs with alleles that were of minor allele frequency >0.005 and with clear genotype classes evident from our single base extension assay (Orchid Biosciences).
³The unique identifier for those SNPs we found to associated with Overall PC response.
⁴Unique identifier composition of the feature SNP combinations optimally associated with variable Overall PC response, selected from the screen described in the text.

Thirteen SNPs in 7 genes (CYP2C8, CYP3A4, GSTM1, CYP3A7, ESD, MAOB and CYP3A5) were identified that met the following criteria: 1) a delta value greater than 0.23 for the distinction of patients based on Overall PC response; or 2) a delta value greater than 0.22, but was present in a gene that harbored at least two such SNPs (including itself; see Table 5, column 5-“MARKERS”; see, also, Table 9, showing delta values). Alleles for each of these SNP loci were within Hardy-Weinberg proportions, and on a gene by gene basis, the associations were highly specific. For example, GSTM1 harbored 3 SNPs associated with response, whereas none was found in related genes such as GSTM3, GSTP1, GSTT1 or GSTT2. Similarly, 4 SNPs were found for CYP2C8, whereas none was found in the related CYP2C9, CYP2C18 and CYP2C19 genes.

Each of the CYP3 genes tested harbored SNPs associated with response using the selected delta value criteria: CYP3A4 (2), CYP3A5 (2) and CYP3A7 (3). No SNP that was associated with paclitaxel response was found in the other 12 cytochrome P450 genes tested. Furthermore, it was not the case that the likelihood of finding a SNP with a good delta value in a gene was a function of how many SNPs were surveyed in that gene. For example, of 63 candidate SNPs tested in the CYP2D6 gene, none was identified as associated with PC response, whereas, of only 49 candidate SNPs tested in the CYP2C8 gene, 4 were identified (Table 5). SNPs that did not meet the selection criteria, but otherwise had reasonable delta values, usually had minor allele frequencies that were low for statistical assurance with this sample.

For each gene with selected SNPs, the association of phase-known alleles with response was tested using an F-statistic and Exact test of population differentiation (Raymond and Rousset, GenePop. Ver. 3.0, Institut des Siences de l'Evolution, 1994; Wier and Cockerham, Evolution 38:1358-1370, 1984, each of which is incorporated herein by reference). SNP combinations of potential interest were defined, and phase-known genotypes (diploid haplotype pairs, hereinafter “multilocus genotype”) were inferred for statistical association with Overall PC response. For example, alleles of 4 CYP2C8 SNPs were identified as associated with Overall PC response, and these 4 SNPs can combine to make 6 different 2-locus combinations. Each of these combinations has its own set of observed phase-known (haplotype) alleles, which can be reconstructed using statistical algorithms (Stephens et al., supra, 2001) and tested for association with response.

Having tested all of the within-gene, phase-known alleles of SNP combinations shown in Table 5, the combination with the lowest p-value was selected for each gene. For genes with only one SNP, all possible 2-locus combinations within that gene, and involving that SNP and other validated SNPs in that gene, whether it had a good delta value or not, were tested; the combination with the lowest p-value was selected (see, for example, Table 5, marker 247 of CYP3A5). The resulting list of SNP combinations included 14 SNPs (Table 5, column 6-“SELECTED COMBOS”).

The multilocus genotypes most strongly associated with variable PC response were those of the CYP2C8, CYP3A4 and ESD genes, and the multilocus genotypes for the GSTM1, CYP3A7 and MAOB genes also were significant (Table 6). The selected combinations for each of these 6 genes are referred to as “feature SNP combinations”, and their alleles are referred to as “genetic features” of variable PC response. Though CYP3A5 multilocus genotypes were not significantly associated with variable PC response at the p=0.05 level, chi-square adjusted residuals on the GT/GT genotype were significantly associated (p=0.033) and, therefore, the selected CYP3A5 combination were included as a feature SNP combination (Table 7G).

TABLE 6
Effect statistics for the ability of multilocus genotypes of selected feature
SNP combinations to resolve between TC responders and non-responders.

| | | | | | Sample |
|---|---|---|---|---|---|
| Gene* | Marker 1 | Marker 2 | F Test P | Exact Test P | N** |
| 1. CYP2C8 | 265 | 371 | <0.000 | <0.000 | 27 |
| 2. CYP3A4 | 664803 | 869772 | <0.000 | 0.014 | 25 |
| 3. ESD | 677 | 648 | 0.012 | 0.019 | 35 |
| 4. GSTM1 | 527 | 200 | 0.018 | 0.019 | 28 |
| 5. CYP3A7 | 121 | 113 | 0.018 | 0.026 | 17 |
| 6. MAOB | 465 | 477 | 0.045 | 0.105 | 38 |
| 7. CYP3A5 | 537 | 247 | 0.072 | 0.082 | 28 |

*Ranked in order from most strongly associated to least associated, using Overall TC response criteria as described in the text. Samples with missing or ambiguous data were eliminated from the analysis.
**individuals with missing or ambiguous genotypes were eliminated from this particular analysis (see methods).

For each of the feature SNP combinations, the associations between haplotype allele combinations and variable PC response were robust using at least one test, and readily apparent using a variety of response criteria, including 5%, 10%, 15%, and 20% Average CA125 reductions, and Overall CA125 reductions. The associations also were statistically significant on the level of the individual haplotype, rather than the diploid pair, and when individual response counts, rather than Average CA125 response, were tabulated. Multiple regression analysis using the CYP3A4 genotypes as explanatory variables (one of the genotypes is merged with the intercept to avoid multi-co-linearity problems) revealed an R-square=0.746, indicating that CYP3A4 genotypes explained 74.6% of the variations in Average PC response. Multiple regression analysis using the twelve genotypes of CYP2C8 as explanatory variables revealed an R-square=0.206, indicating that TAX2C81226 haplotype alleles explain 20.6% of the variability in Average PC response. Multiple Regression analysis of the 20 multilocus genotypes of both CYP3A4 and CYP2C8 revealed that they combined to explain 80% of the variations in Average PC response, and that the remaining 15-20% were explained by genotypes for the other 5 genes.

From counts of the observed multilocus genotypes in Overall PC responders and non-responders, several simple association rules were constructed (Table 7). The CYP3A4 CT/CT multilocus genotype was associated with response with a p-value less than 0.001 (Table 7B), and certain genotypes for CYP3A7 (TG/TG, p=0.002; Table 7D), ESD (TC/TC or CC/CC, p=0.003; Table 7E) and CYP3A5 (GT/GT, p=0.033; Table 7G) also were significantly associated with Overall PC response. In contrast, multilocus genotypes for the CYP2C8 gene (genotypes without a GG or GA haplotype, p=0.033; Table 7A), the GSTM1 gene (genotypes without a PC haplotype, p=0.085; Table 7C) and MAOB gene (genotypes without a CC haplotype, p=0.012; Table 7F) were, to greater or lesser extents, associated with Overall PC non-response. For example, only half of the patients with the MAOB “CC” haplotype are responders (15/28; Table 7F); whereas 87% of the patients lacking the MAOB “CC” haplotype are responders (13/14; Table 7F). As such, a preliminary classification rule can be made, as follows: individuals lacking a copy of the MAOB CC haplotype are (likely to be) responders, and those with at least one copy of the CC haplotype are (likely to be) non-responders. Similar rules can be constructed for each of the other feature SNP combinations.

TABLE 7
Multilocus genotype counts for each of the feature SNP combinations
described in Table 2 using the Overall TC response criteria.
Table 7A

| | | | NON- |
|---|---|---|---|
| CYP2C8 | | RESPONDERS | RESPONDERS |
| G11 | AA/AA | 5 | 1 |
| G12 | AA/AG | 4 | 1 |
| G13 | GA/GA | 8 | 5 |
| G14 | GA/AA | 4 | 1 |
| G15 | AG/AG | 1 | 0 |
| G16 | GA/AG | 1 | 0 |
| G17 | GG/GG | 0 | 4 |
| G18 | GG/GA | 0 | 2 |
| | GG/— OR GA/— | 13 | 12 |
| | OTHER* | 10 | 2 |

* adjusted residual p-value for association with response = 0.033.

TABLE 7B

| | | |
|---|---|---|
| | | NON- |
| CYP3A4 | RESPONDERS | RESPONDERS |

| | | | |
|---|---|---|---|
| G21 | CT/CT | 26 | 7 |
| G22 | CT/CG | 1 | 4 |
| G23 | CT/TG | 0 | 2 |
| G24 | CG/TG | 0 | 2 |
| | CT/CT | 26 | 7 |
| | OTHER* | 1 | 8 |

* adjusted residual p-value for association with non-response <0.001.

TABLE 7C

| | | |
|---|---|---|
| | | NON- |
| GSTM1 | RESPONDERS | RESPONDERS |

| | | | |
|---|---|---|---|
| G31 | CC/CC | 10 | 4 |
| G32 | CC/CT | 2 | 0 |
| G33 | TC/CC | 4 | 3 |
| G34 | TC/TC | 10 | 8 |
| G35 | CT/TC | 0 | 1 |
| | TC/— | 14 | 12 |
| | OTHER* | 12 | 4 |

* adjusted residual p-value for association with response = 0.085.

TABLE 7D

| | | |
|---|---|---|
| | | NON- |
| CYP3A7 | RESPONDERS | RESPONDERS |

| | | | |
|---|---|---|---|
| G41 | TG/TG | 18 | 6 |
| G42 | CC/TG | 4 | 4 |
| G43 | CC/CC | 1 | 3 |
| G44 | TG/TC | 2 | 1 |
| G45 | TG/CG | 0 | 1 |
| | TG/TG* | 18 | 6 |
| | OTHER | 7 | 9 |

* adjusted residual p-value for association with response = 0.023.

TABLE 7E

| | | |
|---|---|---|
| | | NON- |
| ESD | RESPONDERS | RESPONDERS |

| | | | |
|---|---|---|---|
| G51 | CG/CG | 1 | 1 |
| G52 | TC/TC | 10 | 3 |
| G53 | CC/CC | 7 | 1 |
| G54 | TG/TG | 4 | 5 |
| G55 | CC/TC | 0 | 3 |
| | TC/TC or CC/CC* | 17 | 4 |
| | OTHER | 5 | 9 |

* adjusted residual p-value for association with response = 0.003.

TABLE 7F

| | | |
|---|---|---|
| | | NON- |
| MAOB | RESPONDERS | RESPONDERS |

| | | | |
|---|---|---|---|
| G61 | TC/TC | 12 | 2 |
| G62 | CC/TC | 8 | 1 |
| G63 | CC/CC | 6 | 5 |
| G64 | TT/CC | 1 | 0 |
| G65 | TT/TC | 1 | 0 |
| G66 | TC/CC | 0 | 7 |
| | CC/— | 15 | 13 |
| | OTHER* | 13 | 2 |

* adjusted residual p-value for association with response = 0.016.

TABLE 7G

| | | |
|---|---|---|
| | | NON |
| CYP3A5 | RESPONDERS | RESPONDERS |

| | | | |
|---|---|---|---|
| G71 | GT/GT | 17 | 5 |
| G72 | GT/AC | 5 | 4 |
| G73 | GT/AT | 2 | 3 |
| G74 | GT/GC | 2 | 0 |
| G75 | AC/AC | 1 | 2 |
| G76 | AC/GC | 0 | 1 |
| | GT/GT* | 17 | 5 |
| | OTHER | 10 | 10 |

* adjusted residual p-value for association with response = 0.033.

The response or non-response genotypes were unlinked within individuals. Thus, one of the non-responders had a non-response CYP2C8, GSTM1 and MAOB genotype, and also had a response CYP3A7, and ESD multilocus genotype. Another patient, who was a responder, had a non-responder multilocus genotype for CYP2C8, GSTM1, CYP3A7, ESD and MAOB genes, and a responder genotype for CYP3A4 and CYP3A5 genes. Inspection of the rules derived from all of the feature SNP associations suggested that none was of adequate strength to serve as an independent classifier for Overall PC responses. This results suggests that PC response is a complex (i.e. multifactorial) trait.

Feature Modeling

Though specific sequences associated with Average PC response were identified, the results were not necessarily instructive as to how these sequences could be used to comprehensively predict PC response in ovarian cancer patients. Accordingly, a linear discriminate method was used to determine whether a complex genetic model incorporating the genetic features could resolve responders from non-responders. Individual samples (patients) were encoded as n-dimensional vectors (where n=number of observed SNP, haplotype or multilocus genotype alleles). The vectors were used to compute a pooled variance-covariance matrix, the distance between each individual vector and the population mean vectors (patient and responder or non-responder groups, respectively) was calculated, then the sample was binned into the population for which its distance was lowest (Table 8).

TABLE 8
Responder classification probability table constructed by applying the
linear classification technique to allele data from the SNPs (A),
haplotypes (B) and multilocus genotypes (C), as described in the text. The
column is read as follows: Using the 14 SNPs described in the text (A),
non-responders (Row 1, Column 1) were correctly classified as
non-responders using the Overall TC response criteria with a probability
of 0.867 (Row 1, Column 2) and were incorrectly classified as responders
(Row 1, Column 3) with a probability of 0.133.

| | | | |
|---|---|---|---|
| From Actual | Into | | |
| Category | Non Response Class | Into Response Class | Total |

TABLE 8A
14 SNPs in 7 genes

| | | | |
|---|---|---|---|
| Non Response | 86.7% (13) | 13.3% (2) | 100% (15) |
| Response | 11.1% (3) | 88.9% (24) | 100% (27) |
| Total | 38.1% (16) | 61.9% (26) | 100% (42) |

TABLE 8B
22 haplotypes in 7 genes

| | | | |
|---|---|---|---|
| Non Response | 93.3% (14) | 6.7% (1) | 100% (15) |
| Response | 3.7% (1) | 96.3% (26) | 100% (27) |
| Total | 35.7% (15) | 64.3% (27) | 100% (42) |

TABLE 8C
39 multilocus genotypes in 7 genes

| | | | |
|---|---|---|---|
| Non Response | 93.3% (14) | 6.7% (1) | 100% (15) |
| Response | 0 (0) | 100% (27) | 100% (27) |
| Total | 38.1% (14) | 61.9% (28) | 100% (42) |

Using SNP genotypes for the 5 genes with the strongest associations (CYP2C8, CYP3A4, GSTM1, CYP3A7, ESD), the corrected probability for partitioning responders and non-responders into the correct group was 85.7% (test efficiency, 36/42). Using SNP genotypes for all 7 of the associated genes, rather than just the 5 strongest, an 88.1% test efficiency was obtained for partitioning responders and non-responders into the correct group (37/42; see Table 8A). A substantially improved corrected test efficiency of 95.2% (40/42) was achieved using haplotypes for each of the 7 genes, rather than SNPs (Table 8B), and use of the multilocus genotypes (diploid pairs of haplotypes) for each of the 7 genes generated a corrected test efficiency of 97.6% (41/42) for partitioning responders and non-responders into the correct group (Table 8C).

Monte Carlo simulation of multilocus genotypes with 10,000 iterations produced a mean corrected test efficiency of 97% (in agreement with the observed data), a median of 98%, a standard deviation of 4.6% and a 95% CI of (0.94, 1.0). Using the non-response classification as a positive test result, this classifier revealed a non-response sensitivity of 93%, a response specificity of 100%, a predictive value for the negative (non-responder) inference of 100%, and a predictive value for the positive (responder) inference of 96.4%. The results were not significantly different when using a quadratic rather than the linear classification technique, nor were they significantly different using the Average PC response criteria (using a 10% and a 15% reduction threshold).

TABLE 9

| Gene | Marker | DELTA | EAE |
|---|---|---|---|
| CYP2C8E8_92_GA (1) | 265 | 0.3571 | 0.27791 |
| CYP3A4 (3) | 664803 | 0.3386 | 0.49744 |
| CYP3A4_RS2687117_TC (16) | 232 | 0.327 | 0.6307 |
| CYP3A7RS2037498_TC (9) | 121 | 0.3194 | 0.239 |
| CYP3A7RS2687075_TC (17) | 229 | 0.3125 | 0.19901 |
| ESD1216961_TC (5) | 677 | 0.303 | 0.16487 |
| GSTM1421547_TC (7) | 527 | 0.2853 | 0.14835 |
| ASIP8818ag_GA (30) | 859 | 0.2833 | 0.15162 |
| CYP2C8_RS1891070_GA (15) | 357 | 0.2708 | 0.15974 |
| CYP3A7RS2222411_GC (10) | 113 | 0.2633 | 0.16006 |
| CYP2C8_RS947172_GA (3) | 371 | 0.2564 | 0.14468 |
| CYP3A5776746_GA (13) | 537 | 0.246 | 0.14316 |
| CYP2C8_RS1891071_GA (35) | 369 | 0.2361 | 0.11401 |
| MAOB1799836_TC (11) | 465 | 0.2321 | 0.09539 |
| TUBB42302897_GA (18) | 761 | 0.2267 | 0.21327 |
| CYP2C9RS1934969_TA (19) | 39 | 0.2208 | 0.08842 |
| TYRP (20) | 886938 | 0.2186 | 0.08615 |
| CYP3A7RS2687145_TC (21) | 140 | 0.2185 | 0.11413 |
| ASIP819136_GA (22) | 559 | 0.2173 | 0.13253 |
| CYP2D6_RS769261_GT (23) | 423 | 0.2172 | 0.09438 |
| AIM35397_GT (24) | 928 | 0.2167 | 0.09081 |
| GSTT22267047_TC (25) | 464 | 0.2074 | 0.09112 |
| DCT2892681_GC (26) | 650 | 0.2074 | 0.08254 |
| DCT2296498_GA (27) | 701 | 0.2074 | 0.19985 |
| OCA2 (28) | 217458 | 0.2054 | 0.07484 |
| CYP4B1RS751028_GA (29) | 292 | 0.2048 | 0.08898 |
| ESD1216956_GC (6) | 648 | 0.2037 | 0.07652 |
| POR17685_GA (31) | 691 | 0.2 | 0.10238 |
| ASIP2424987_GA (32) | 861 | 0.2 | 0.09232 |
| RAB272444039_GT (33) | 925 | 0.2 | 0.07044 |
| AIM35389_GA | 903 | 0.1994 | 0.08013 |
| OCA2 | 712052 | 0.1939 | 0.07603 |
| ASIP819135_GA | 552 | 0.1933 | 0.06614 |
| CYP2D6 | 869785 | 0.192 | 0.54798 |
| GSTA22180315_TC | 500 | 0.1889 | 0.07664 |
| GSTT2678863_GA | 786 | 0.1889 | 0.06314 |
| OCA2 | 217455 | 0.1875 | 0.069 |
| CYP1A2_RS2960193_TC | 182 | 0.1875 | 0.0642 |
| CYP3A5RS1419745_GA | 288 | 0.1875 | 0.0642 |
| SILV1052206_TC | 656 | 0.1875 | 0.07138 |
| CYP3A7RS2687134_CA | 59 | 0.1868 | 0.09034 |
| CYP2C8E93UTR_221_TC | 155 | 0.1852 | 0.08493 |
| CYP2C8_RS1058932_TC | 164 | 0.1852 | 0.08493 |
| CYP3A7RS2687133_GA | 379 | 0.1852 | 0.08493 |
| GSTA22608677_GC | 451 | 0.1852 | 0.08031 |
| HMGCR | 664797 | 0.1846 | 0.09538 |
| POR8509_GA | 689 | 0.1845 | 0.0721 |
| CYP3A7RS2687143_GA | 364 | 0.1815 | 0.0737 |
| TUBB412207_GA | 791 | 0.1815 | 0.20366 |
| OCA2 | 886894 | 0.1811 | 0.05765 |
| CYP3A7RS2687140_GA | 287 | 0.181 | 0.06151 |
| GSTT2140186_GA | 545 | 0.18 | 0.05693 |
| ACE_1987693_TC | 163 | 0.1781 | 0.47034 |
| HMGCS1 | 886900 | 0.1769 | 0.05499 |
| MYO5A2242057_TC | 896 | 0.1754 | 0.05408 |
| GSTP12370143_TC | 533 | 0.1741 | 0.05909 |
| MYO5A1724638_GC | 813 | 0.1733 | 0.07265 |
| CYP1B1RS1056837_TC | 151 | 0.1731 | 0.06122 |
| ESD9778_GA | 684 | 0.1731 | 0.1042 |
| MYO5A1693510_GT | 941 | .1682 | 0.07103 |
| CYP3A7 | 712033 | 0.1679 | 0.05842 |
| CYP2D6 | 554370 | 0.1663 | 0.45126 |
| CYP2C191799853_TC | 491 | 0.1652 | 0.35789 |
| FDPS | 869769 | 0.163 | 0.07126 |
| ASIP2424984_TC | 468 | 0.163 | 0.05781 |
| CYP2E1RS2854140_GA | 358 | 0.1625 | 0.0598 |
| GSTT21803690_TC | 496 | 0.1624 | 0.07627 |
| CYP3A5RS15524_TC (14) | 247 | 0.1614 | 0.06387 |
| CYP3A515524_TC | 526 | 0.1603 | 0.06061 |
| TUBB1054332_GA | 763 | 0.16 | 0.04609 |
| MYO5A1724606_GT | 939 | 0.16 | 0.06726 |
| GSTA21051536_GC | 440 | 0.1591 | 0.0505 |
| GSTT22267048_TC | 492 | 0.1556 | 0.04983 |
| SILV1132095_GT | 704 | 0.154 | 0.09113 |
| CYP3A4 (4) | 869772 | 0.1535 | 0.42037 |
| MYO5A752864_TC | 835 | 0.1533 | 0.04149 |
| MYO5A935892_GA | 898 | 0.1533 | 0.04149 |
| CYP2C181799853_TC | 490 | 0.1528 | 0.34279 |
| MYO5A1724639_TC | 843 | 0.1522 | 0.04154 |
| CYP2C8E2E3_397_TC | 134 | 0.15 | 0.04515 |
| ABC117064_TA | 630 | 0.15 | 0.07157 |

Numbers in parentheses in Column 1 are Sequence Identifiers (SEQ ID NO:)

The disclosed classifiers likely are predictive of variable paclitaxel rather than carboplatin metabolism because carboplatin is relatively stable and excreted largely in unchanged form in the urine. In contrast, paclitaxel is a highly aromatic molecule that is extensively metabolized and excreted through the urine and feces in a highly oxidized form (see, for example, McFadyen et al., Biochem. Pharmacol. 62:207-212, 2001). Further, the metabolism and clearance pharmacokinetics are widely variable between paclitaxel patients. In vitro studies suggested that paclitaxel is metabolized by CYP2C8 (Dai et al., Pharmacogenetics 11:597-607, 2001; Soyama et al., Biol. Pharm. Bull. 24:1427-1430, 2001) and to a lesser extent by CYP3A4 (Baumhakel et al., Int. J. Clin. Pharmacol. Ther. 39:517-528, 2001). However, prior to the present disclosure, it was not clear whether variation in sequence for these genes, or perhaps other genes as well, was related to variable PC response in vivo.

Because patients are chemotherapy naive prior to first line treatment, their tumors are not likely to have developed resistance. As such, variable first line PC response was presumed to be a pharmacokinetic phenomena rather than a tumor resistance phenomena. As such, a pan-xenobiotic metabolism gene study was initiated, testing all of the SNPs that could be identified in each of these genes. No assumption was made as to SNPs to be examined or the genes that would be associated. As disclosed herein, the associated SNPs clustered within certain genes, indicating that the identified markers are of biological significance. In particular, identification of the CYP3A4 and CYP2C8 genes as the most strongly associated with PC response is meaningful, in view of studies implicating these genes in paclitaxel metabolism (Baumhakel et al., supra, 2001).

In addition to CYP3A4 and CYP2C8, 5 other genes with associated SNP sequences were identified as strongly associated with PC response. These genes include alleles of the ESD-D (ESD) gene. Although the ESD gene has not previously been reported to be relevant with respect to paclitaxel metabolism, paclitaxel contains 5 ester groups and, therefore, may be a substrate for the ESD gene product. Similarly, the MAOB gene has not previously been associated with paclitaxel response. The relevance, if any, of this gene to paclitaxel metabolism is not clear because paclitaxel does not contain an amine group, though it does contain an amide group. However; the association of the MAOB gene with variable PC response can be due to involvement with carboplatin metabolism or through some other expression products via an indirect means. Another associated gene, GSTM1, encodes glutathione S transferase (GST), which previously has been identified as involved in taxane metabolism; the expression level of GST has been correlated with the IC50 for docetaxel (Park et al., Int. J. Oncol. 20:333-338, 2002; Masanek et al., Anticancer Drugs 8:189-198, 1997), which is a related compound subject to the same metabolic machinery as paclitaxel (Desai et al., Eur. J. Drug Metab. Pharmacokinet. 23:417424, 1998).

Alleles for all three of the CYP3 family members tested were associated with PC response and, further, the presence of the associated alleles for each of the three CYP3 family members was associated with response rather than non-response. This result is interesting because the CYP3A family members are coordinately regulated through the pregnane X receptor via induction by a wide variety of xenobiotic compounds, and because various CYP3A gene products are known to act on common substrates (reviewed by Quattrochi and Guzelian, Drug Metab. Dispos. 29:615-622, 2001; Ripp et al., Drug Metab. Dispos. 30:570-575, 2002). The identification of PC response associations in multiple CYP3A genes is consistent with previous reports on the concerted action of these genes, and provides support that the disclosed associations are biologically significant.

Whether the SNP loci disclosed herein are linked with previously defined variants is not yet known. Using in vitro assays, in which cells were contacted in culture with paclitaxel, two paclitaxel-incompetent CYP2C8 haplotype alleles have been described, including the CYP2C8*2 haplotype, which was found only in African-Americans, and the CYP2C8*3 haplotype, which was found primarily in Caucasians (Dai et al., Pharmacogenetics 11:597-607, 2001). However, the relevance of these haplotypes to paclitaxel responsiveness in vivo has not been determined, and the SNP loci of the CYCP2C8*2 and CYP2C8*3 haplotypes were not identified in the present study. The SNP loci disclosed herein have not been incorporated in the existing CYP2C8 or CYP3A4 nomenclature, which suggests that these nomenclature systems are incomplete, and none has been described as relating to responsiveness of paclitaxel or any other drug. However, it some or all of the disclosed CYP2C8 alleles may be linked to the two coding changes that constitute the CYP2C8*3 variant (ARG139LYS and LYS399ARG). Nonetheless, the disclosed haplotypes are part of the natural distribution of variants in the human population, and whether or how they are linked to previously defined variants is of more theoretical than practical importance. As the first complex genetic model yet described for predicting an individual's proclivity to respond to PC therapy, the disclosed classifier represents a first step towards pharmacogenetic manipulation of first line chemotherapy response rates.

Although the invention has been described with reference to the above examples, it will be understood that modifications and variations are encompassed within the spirit and scope of the invention. Accordingly, the invention is limited only by the following claims.

What is claimed is:

1. A method for inferring responsiveness of a subject to paclitaxel treatment from a nucleic acid sample of the subject, comprising detecting, in the nucleic acid sample, a single nucleotide polymorphism (SNP) associated with paclitaxel responsiveness, wherein the SNP comprises:
a) nucleotides of a cytochrome P450 (CYP2C8) gene, comprising a nucleotide corresponding to nucleotide 83 of SEQ ID NO:1, nucleotide 251 of SEQ ID NO:2; nucleotide 181 of SEQ ID NO:15, or nucleotide 75 of SEQ ID NO:35;
b) nucleotides of a CYP3A4 gene, comprising a nucleotide corresponding to nucleotide 401 of SEQ ID NO:3, nucleotide 437 of SEQ ID NO:4; nucleotide 151 of SEQ ID NO:16, or nucleotide 1466 of SEQ ID NO:34;
c) nucleotides of an esterase D (ESD) gene, comprising a nucleotide corresponding to nucleotide 702 of SEQ ID NO:5, or nucleotide 201 of SEQ ID NO:6;
d) nucleotides of a glutathione S-transferase (GSTM1) gene, comprising a nucleotide corresponding to nucleotide 201 of SEQ ID NO:7, or nucleotide 191 of SEQ ID NO:8;
e) nucleotides of a CYP3A7 gene, comprising a nucleotide corresponding to nucleotide 401 of SEQ ID NO:9, nucleotide 541 of SEQ ID NO:10, nucleotide 151 of SEQ ID NO:17, or nucleotide 201 of SEQ ID NO:21;
f) nucleotides of a monoamine oxidase (MAOB) gene, comprising a nucleotide corresponding to nucleotide 501 of SEQ ID NO:11, or nucleotide 60 of SEQ ID NO:12;
g) nucleotides of a CYP3A5 gene, comprising a nucleotide corresponding to nucleotide 201 of SEQ ID NO:13, or nucleotide 101 of SEQ ID NO:14;
h) nucleotides of an agouti signaling protein (ASIP) gene, comprising a nucleotide corresponding to nucleotide 201 of SEQ ID NO:22, nucleotide 26 of SEQ ID NO:30, or nucleotide 101 of SEQ ID NO:32;
i) nucleotides of a tubulin (TUBB) gene, comprising a nucleotide corresponding to nucleotide 61 of SEQ ID NO:18;
j) nucleotides of a CYP2C9 gene, comprising a nucleotide corresponding to nucleotide 122 of SEQ ID NO:19;
k) nucleotides of a tyrosinase-related protein (TYRP) gene, comprising a nucleotide corresponding to nucleotide 592 of SEQ ID NO:20;
l) nucleotides of a CYP2D6 gene, comprising a nucleotide corresponding to nucleotide 201 of SEQ ID NO:23;
m) nucleotides of an AIM gene, comprising nucleotide 201 of SEQ ID NO₂₄;
n) nucleotides of a GSTT gene, comprising a nucleotide corresponding to nucleotide 61 of SEQ ID NO:25;
o) nucleotides of a dopachrome tautomerase (D CT) gene, comprising a nucleotide corresponding to nucleotide 201 of SEQ ID NO:26, or nucleotide 61 of SEQ ID NO:27;
p) nucleotides of an oculocutaneous albinism (OCA) gene, comprising a nucleotide corresponding to nucleotide 135 of SEQ ID NO:28;
q) nucleotides of a CYP4B gene, comprising a nucleotide corresponding to nucleotide 123 of SEQ ID NO:29;
r) nucleotides of a POR gene, comprising a nucleotide corresponding to nucleotide 61 of SEQ ID NO:31;
s) nucleotides of an RAB gene, comprising a nucleotide corresponding to nucleotide 201 of SEQ ID NO:33; or
t) a combination of SNPs comprising nucleotides as set forth in a) through s),
wherein the SNP or combination of SNPs indicates that the subject is a paclitaxel responder or that the subject is a paclitaxel non-responder, thereby inferring responsiveness of the subject to paclitaxel treatment.

2. The method of claim 1, wherein the SNP comprises nucleotides as set forth in a) through g), or a combination thereof.

3. The method of claim 1, wherein the SNP comprises nucleotide 83 of SEQ ID NO:1, nucleotide 251 of SEQ ID NO:2, nucleotide 401 of SEQ ID NO:3, nucleotide 702 of SEQ ID NO:5, nucleotide 201 of SEQ ID NO:6, nucleotide 201 of SEQ ID NO:7, nucleotide 191 of SEQ ID NO:8, nucleotide 401 of SEQ ID NO:9, nucleotide 541 of SEQ ID NO:10, nucleotide 501 of SEQ ID NO:11, nucleotide 60 of SEQ ID NO:12, nucleotide 201 of SEQ ID NO:13, nucleotide 101 of SEQ ID NO:14, nucleotide 181 of SEQ ID NO:15, nucleotide 151 of SEQ ID NO:16, nucleotide 151 of SEQ ID NO:17, nucleotide 75 of SEQ ID NO:35, or a combination thereof.

4. The method of claim 1, wherein the SNP comprises nucleotide 83 of SEQ ID NO:1, nucleotide 251 of SEQ ID NO:2, nucleotide 401 of SEQ ID NO:3, nucleotide 437 of SEQ ID NO:4, nucleotide 702 of SEQ ID NO:5, nucleotide 201 of SEQ ID NO:6, nucleotide 201 of SEQ ID NO:7, nucleotide 191 of SEQ ID NO:8, nucleotide 401 of SEQ ID NO:9, nucleotide 541 of SEQ ID NO:10, nucleotide 501 of SEQ ID NO:11, nucleotide 60 of SEQ ID NO:12, nucleotide 201 of SEQ ID NO:13, nucleotide 101 of SEQ ID NO:14, nucleotide 181 of SEQ ID NO:15, nucleotide 151 of SEQ ID NO:16, nucleotide 151 of SEQ ID NO:17, nucleotide 61 of SEQ ID NO:18, nucleotide 122 of SEQ ID NO:19, nucleotide 26 of SEQ ID NO:30, nucleotide 75 of SEQ ID NO:35, or a combination thereof.

5. The method of claim 1, wherein the SNP or combination of SNPs indicates that the subject is a paclitaxel responder.

6. The method of claim 5, wherein nucleotide 83 of SEQ ID NO:1 is A; nucleotide 401 of SEQ ID NO:3 is C; nucleotide 437 of SEQ ID NO:4 is T; nucleotide 201 of SEQ ID NO:6 is C; nucleotide 201 of SEQ ID NO:7 is C; nucleotide 401 of SEQ ID NO:9 is T; nucleotide 541 of SEQ ID NO:10 is G; nucleotide 501 of SEQ ID NO:11 is T; nucleotide 60 of SEQ ID NO:12 is T; nucleotide 201 of SEQ ID NO:13 is G; nucleotide 101 of SEQ ID NO:14 is T; nucleotide 181 of SEQ ID NO:15 is G; nucleotide 151 of SEQ ID NO:16 is C; nucleotide 151 of SEQ ID NO:17 is C; nucleotide 61 of SEQ ID NO:18 is G; nucleotide 122 of SEQ ID NO:19 is A; nucleotide 26 of SEQ ID NO:30 is A; nucleotide 75 of SEQ ID NO:35 is G; or a combination thereof.

7. The method of claim 1, wherein the SNP or combination of SNPs indicates that the subject is a paclitaxel non-responder.

8. The method of claim 7, wherein nucleotide 83 of SEQ ID NO:1 is G; nucleotide 401 of SEQ ID NO:3 is T; nucleotide 437 of SEQ ID NO:4 is G; nucleotide 201 of SEQ ID NO:6 is G; nucleotide 201 of SEQ ID NO:7 is T; nucleotide 401 of SEQ ID NO:9 is C; nucleotide 541 of SEQ ID NO:10 is C; nucleotide 501 of SEQ ID NO:11 is C; nucleotide 60 of SEQ ID NO:12 is C; nucleotide 201 of SEQ ID NO:13 is A; nucleotide 101 of SEQ ID NO:14 is C; nucleotide 181 of SEQ ID NO:15 is A; nucleotide 151 of SEQ ID NO:16 is T; nucleotide 151 of SEQ ID NO:17 is T; nucleotide 61 of SEQ ID NO:18 is A; nucleotide 122 of SEQ ID NO:19 is T; nucleotide 26 of SEQ ID NO:30 is G; nucleotide 75 of SEQ ID NO:35 is A; or a combination thereof.

9. The method of claim 1, wherein detecting a SNP associated with paclitaxel responsiveness comprises contacting the nucleic acid sample with an oligonucleotide probe that selectively hybridizes to a nucleotide sequence comprising the SNP, or a nucleotide sequence complementary thereto, and detecting selective hybridization of the oligonucleotide probe.

10. The method of claim 9, wherein the oligonucleotide probe comprises a detectable label, and wherein detecting selective hybridization of the probe comprises detecting the detectable label.

11. The method of claim 10, wherein the detectable label comprises a fluorescent label, a luminescent label, a radionuclide, or a chemiluminescent label.

12. The method of claim 9, wherein the oligonucleotide comprises a bilabeled oligonucleotide probe, comprising a fluorescent moiety and a fluorescent quencher.

13. The method of claim 1, wherein detecting a SNP associated with paclitaxel responsiveness comprises
contacting the nucleic acid sample with at least a first oligonucleotide primer, which selectively hybridizes to a nucleotide sequence 3′ to and flanking the SNP position, or a nucleotide sequence complementary thereto, under conditions sufficient for extension of the primer by a polymerase, and
detecting whether a primer extension product is generated, wherein the presence or absence of the primer extension product is indicative of a nucleotide at the SNP position.

14. The method of claim 1, wherein detecting a SNP associated with paclitaxel responsiveness comprises
contacting the nucleic acid sample with at least a first amplification primer pair, under conditions suitable for generating an amplification product comprising the SNP position; and
detecting whether a nucleotide occurrence of the SNP in the amplification product is associated with a paclitaxel responder or a paclitaxel non-responder.

15. The method of claim 14, wherein the nucleotide occurrence is detected by sequencing the amplification product.

16. The method of claim 14, wherein the nucleotide occurrence is detected using an oligonucleotide probe specific for the nucleotide occurrence.

17. A method for inferring responsiveness of a subject to paclitaxel treatment from a nucleic acid sample of the subject, the method comprising detecting in the nucleic acid sample, a haplotype allele associated with paclitaxel responsiveness, wherein the haplotype comprises:
a) nucleotides of a CYP2C8 gene, comprising nucleotides corresponding to at least two of nucleotide 83 of SEQ ID NO:1, nucleotide 251 of SEQ ID NO:2; nucleotide 181 of SEQ ID NO:15, and nucleotide 75 of SEQ ID NO:35;
b) nucleotides of a CYP3A4 gene, comprising nucleotides corresponding to at least two of nucleotide 401 of SEQ ID NO:3, nucleotide 437 of SEQ ID NO:4; nucleotide 151 of SEQ ID NO:16, and nucleotide 1466 of SEQ ID NO:34;
c) nucleotides of an ESD gene, comprising nucleotides corresponding to at least nucleotide 702 of SEQ ID NO:5, and nucleotide 201 of SEQ ID NO:6;
d) nucleotides of a GSTM1 gene, comprising nucleotides corresponding to at least nucleotide 201 of SEQ ID NO:7, and nucleotide 191 of SEQ ID NO:8;
e) nucleotides of a CYP3A7 gene, comprising nucleotides corresponding to at least two of nucleotide 401 of SEQ ID NO:9, nucleotide 541 of SEQ ID NO:10, nucleotide 151 of SEQ ID NO:17, and nucleotide 201 of SEQ ID NO:21;
f) nucleotides of a MAOB gene, comprising nucleotides corresponding to at least nucleotide 501 of SEQ ID NO:11, and nucleotide 60 of SEQ ID NO:12;
g) nucleotides of a CYP3A5 gene, comprising nucleotides corresponding to at least nucleotide 201 of SEQ ID NO:13, and nucleotide 101 of SEQ ID NO:14;
h) nucleotides of an ASIP gene, comprising nucleotides corresponding to at least two of nucleotide 201 of SEQ ID NO:22, nucleotide 26 of SEQ ID NO:30, and nucleotide 101 of SEQ ID NO:32;
i) nucleotides of a TUBB gene, comprising nucleotides corresponding to nucleotide 61 of SEQ ID NO:18, and at least a second SNP of a TUBB gene, wherein the SNP is associated with paclitaxel responsiveness;
j) nucleotides of a CYP2C9 gene, comprising nucleotides corresponding to nucleotide 122 of SEQ ID NO:19, and at least a second SNP of a CYP2C9 gene, wherein the SNP is associated with paclitaxel responsiveness;
k) nucleotides of a TYRP gene, comprising nucleotides corresponding to nucleotide 592 of SEQ ID NO:20, and at least a second SNP of a TYRP gene, wherein the SNP is associated with paclitaxel responsiveness;
l) nucleotides of a CYP2D6 gene, comprising nucleotides corresponding to nucleotide 201 of SEQ ID NO:23, and at least a second SNP of a CYP2D6 gene, wherein the SNP is associated with paclitaxel responsiveness;
m) nucleotides of an AIM gene, comprising nucleotides corresponding to nucleotide 201 of SEQ ID NO₂₄, and at least a second SNP of an AIM gene, wherein the SNP is associated with paclitaxel responsiveness;
n) nucleotides of a GSTT gene, comprising nucleotides corresponding to nucleotide 61 of SEQ ID NO:25, and at least a second SNP of a GST gene, wherein the SNP is associated with paclitaxel responsiveness;
o) nucleotides of a DCT gene, comprising nucleotides corresponding to at least nucleotide 201 of SEQ ID NO:26, and nucleotide 61 of SEQ ID NO:27;
p) nucleotides of an OCA gene, comprising nucleotides corresponding to nucleotide 135 of SEQ ID NO:28, and at least a second SNP of a OCA gene, wherein the SNP is associated with paclitaxel responsiveness;
q) nucleotides of a CYP4B gene, comprising nucleotides corresponding to nucleotide 123 of SEQ ID NO:29, and at least a second SNP of a CYP4B gene, wherein the SNP is associated with paclitaxel responsiveness;
r) nucleotides of a POR gene, comprising nucleotide 61 of SEQ ID NO:31, and at least a second SNP of a POR gene, wherein the SNP is associated with paclitaxel responsiveness;
s) nucleotides of an RAB gene, comprising nucleotides corresponding to nucleotide 201 of SEQ ID NO:33, and at least a second SNP of a RAB gene, wherein the SNP is associated with paclitaxel responsiveness; or
t) a combination of haplotype alleles as set forth in a) through s),
wherein the haplotype allele or combination of haplotype alleles indicates that the subject is a paclitaxel responder or that the subject is a paclitaxel non-responder, thereby inferring responsiveness of the subject to paclitaxel treatment.

18. The method of claim 17, wherein the haplotype allele comprises:
a) nucleotides of a CYP2C8 gene, which comprise nucleotide 83 of SEQ ID NO:1, and nucleotide 251 of SEQ ID NO:2;
b) nucleotides of a CYP3A4 gene, which comprise nucleotide 401 of SEQ ID NO:3, and nucleotide 437 of SEQ ID NO:4;
c) nucleotides of an ESD gene, which comprise nucleotide 702 of SEQ ID NO:5, and nucleotide 201 of SEQ ID NO:6;
d) nucleotides of a GSTM1 gene, which comprise nucleotide 201 of SEQ ID NO:7, and nucleotide 191 of SEQ ID NO:8;
e) nucleotides of a CYP3A7 gene, which comprise nucleotide 401 of SEQ ID NO:9, and nucleotide 541 of SEQ ID NO:10;
f) nucleotides of a MAOB gene, which comprise nucleotide 501 of SEQ ID NO:11, and nucleotide 60 of SEQ ID NO:12;
g) nucleotides of a CYP3A5 gene, which comprise nucleotide 201 of SEQ ID NO:13, and nucleotide 101 of SEQ ID NO:14; or
h) a combination of haplotype alleles a) through g).

19. The method of claim 18, wherein the CYP2C8 gene the haplotype allele comprises GG or GA; the CYP3A4 gene the haplotype allele comprises CT; the ESD gene haplotype allele comprises TC or CC; the GSTM1 gene haplotype allele comprises TC; the CYP3A7 gene haplotype allele comprises TG; the MAOB gene haplotype allele comprises CC; or the CYP3A5 gene haplotype allele comprises GT.

20. The method of claim 17, wherein the haplotype allele or combination of haplotype alleles indicates that the subject is a paclitaxel responder.

21. The method of claim 20, wherein the subject has CYP2C8 gene haplotype alleles other than GG or GA; a CYP3A4 gene haplotype allele CT; an ESD gene haplotype allele TC or CC; GSTM1 gene haplotype alleles other than TC; a CYP3A7 gene haplotype allele TG; MAOB gene haplotype alleles other than CC; a CYP3A5 gene haplotype allele GT; or a combination thereof.

22. The method of claim 21, wherein the haplotype allele comprises diploid haplotype alleles, and wherein the subject has CYP2C8 gene diploid haplotype alleles other than GG/NN or GA/NN; CYP3A4 gene diploid haplotype alleles CT/CT; ESD gene diploid haplotype alleles TC/TC or CC/CC; GSTM1 gene diploid haplotype alleles other than TC/NN; CYP3A7 gene diploid haplotype alleles TG/TG; MAOB gene diploid haplotype alleles other than CC/NN; CYP3A5 gene diploid haplotype alleles GT/GT; or a combination thereof.

23. The method of claim 17, wherein the haplotype allele or combination of haplotype alleles indicates that the subject is a paclitaxel non-responder.

24. The method of claim 23, wherein the subject has CYP3A4 gene haplotype alleles other than CT.

25. The method of claim 24, wherein the haplotype allele comprises diploid haplotype alleles, and wherein the subject has CYP3A4 gene diploid haplotype alleles other than CT/CT.

26. A method for inferring responsiveness of a subject to paclitaxel treatment from a nucleic acid sample of the subject, the method comprising detecting in the nucleic acid sample, diploid haplotype alleles associated with paclitaxel responsiveness, wherein the diploid haplotype alleles comprise:
a) nucleotides of a CYP2C8 gene, which comprise nucleotides corresponding to nucleotide 83 of SEQ ID NO:1, and nucleotide 251 of SEQ ID NO:2;
b) nucleotides of a CYP3A4 gene, which comprise nucleotides corresponding to nucleotide 401 of SEQ ID NO:3, and nucleotide 437 of SEQ ID NO:4;
c) nucleotides of an ESD gene, which comprise nucleotides corresponding to nucleotide 702 of SEQ ID NO:5, and nucleotide 201 of SEQ ID NO:6;
d) nucleotides of a GSTM1 gene, which comprise nucleotides corresponding to nucleotide 201 of SEQ ID NO:7, and nucleotide 191 of SEQ ID NO:8;
e) nucleotides of a CYP3A7 gene, which comprise nucleotides corresponding to nucleotide 401 of SEQ ID NO:9, and nucleotide 541 of SEQ ID NO:10;
f) nucleotides of a MAOB gene, which comprise nucleotides corresponding to nucleotide 501 of SEQ ID NO:11, and nucleotide 60 of SEQ ID NO:12;
g) nucleotides of a CYP3A5 gene, which comprise nucleotides corresponding to nucleotide 201 of SEQ ID NO:13, and nucleotide 101 of SEQ ID NO:14; or
h) a combination of diploid haplotype alleles as set forth in a) through g).

27. The method of claim 26, wherein the CYP2C8 gene the diploid haplotype alleles comprise GG/NN or GA/NN; the CYP3A4 gene the diploid haplotype alleles comprise CT/CT; the ESD gene diploid haplotype alleles comprise TC/NN or CC/NN; the GSTM1 gene diploid haplotype alleles comprise TC/NN; the CYP3A7 gene diploid haplotype alleles comprise TG/TG; the MAOB gene diploid haplotype alleles comprise CC/NN; or the CYP3A5 gene diploid haplotype alleles comprise GT/GT.

28. The method of claim 26, wherein the diploid haplotype alleles or combination of diploid haplotype alleles indicates that the subject is a paclitaxel responder.

29. The method of claim 28, wherein the subject has CYP2C8 gene diploid haplotype alleles other than GG/NN or GA/NN; CYP3A4 gene diploid haplotype alleles CT/CT; ESD gene diploid haplotype alleles TC/TC or CC/CC; GSTM1 gene diploid haplotype alleles other than TC/NN; CYP3A7 gene diploid haplotype alleles TG/TG; MAOB gene diploid haplotype alleles other than CC/NN; CYP3A5 gene diploid haplotype alleles GT/GT; or a combination thereof.

30. The method of claim 26, wherein the diploid haplotype allele or combination of diploid haplotype alleles indicates that the subject is a paclitaxel non-responder.

31. The method of claim 30, wherein the subject has CYP3A4 gene diploid haplotype alleles other than CT/CT.

32. An isolated nucleic acid molecule, comprising at least 15 contiguous nucleotides of a polynucleotide as set forth in
SEQ ID NO:1, said at least 15 contiguous nucleotides comprising nucleotide 83 of SEQ ID NO:1, wherein nucleotide 83 is G;
SEQ ID NO:3, said at least 15 contiguous nucleotides comprising nucleotide 401 of SEQ ID NO:3, wherein nucleotide 401 is T;
SEQ ID NO:4, said at least 15 contiguous nucleotides comprising nucleotide 437 of SEQ ID NO:4, wherein nucleotide 437 is G; or
SEQ ID NO:8, said at least 15 contiguous nucleotides comprising nucleotide 191 of SEQ ID NO:8, wherein nucleotide 191 is T,
or an isolated polynucleotide complementary thereto.

33. The isolated nucleic acid molecule of claim 32, which is a polyribonucleotide.

34. A kit, comprising the nucleic acid molecule of claim 32.

35. The kit of claim 34, further comprising at least one oligonucleotide that selectively hybridizes to said nucleic acid molecule, wherein said oligonucleotide selectively hybridizes at or near nucleotide 83 of SEQ ID NO:1; nucleotide 401 of SEQ ID NO:3; nucleotide 437 of SEQ ID NO:4, or nucleotide 191 of SEQ BD NO:8.

36. A plurality of isolated nucleic acid molecules, comprising at least one nucleic acid molecule of claim 32.

37. The plurality of isolated nucleic acid molecules of claim 36, further comprising at least 15 contiguous nucleotides of a polynucleotide as set forth in
SEQ ID NO:1, said at least 15 contiguous nucleotides comprising nucleotide 83 of SEQ ID NO:1, wherein nucleotide 83 is A;
SEQ ID NO:3, said at least 15 contiguous nucleotides comprising nucleotide 401 of SEQ ID NO:3, wherein nucleotide 401 is C;
SEQ ID NO:4, said at least 15 contiguous nucleotides comprising nucleotide 437 of SEQ ID NO:4, wherein nucleotide 437 is T; or
SEQ ID NO:8, said at least 15 contiguous nucleotides comprising nucleotide 191 of SEQ ID NO:8, wherein nucleotide 191 is C,
or a polynucleotide complementary to any of the foregoing.

38. The plurality of isolated nucleic acid molecules of claim 36, further comprising a nucleic acid molecule comprising at least 15 contiguous nucleotides of a polynucleotide comprising nucleotide 251 of SEQ ID NO:2; nucleotide 702 of SEQ ID NO:5; nucleotide 201 of SEQ ID NO:6; nucleotide 201 of SEQ ID NO:7; nucleotide 191 of SEQ ID NO:8; nucleotide 401 of SEQ ID NO:9; nucleotide 541 of SEQ ID NO:10; nucleotide 501 of SEQ ID NO:11; nucleotide 60 of SEQ ID NO:12; nucleotide 201 of SEQ ID NO:13; nucleotide 101 of SEQ ID NO:14; nucleotide 181 of SEQ ID NO:15; nucleotide 151 of SEQ ID NO:16; nucleotide 151 of SEQ ID NO:17; nucleotide 61 of SEQ ID NO:18; nucleotide 122 of SEQ ID NO:19; nucleotide 592 of SEQ ID NO:20; nucleotide 201 of SEQ ID NO:21; nucleotide 201 of SEQ ID NO:22; nucleotide 201 of SEQ ID NO:23; nucleotide 201 of SEQ ID NO:24; nucleotide 61 of SEQ ID NO:25; nucleotide 201 of SEQ ID NO:26, or nucleotide 61 of SEQ ID NO:27; nucleotide 135 of SEQ ID NO:28; nucleotide 123 of SEQ ID NO:29; nucleotide 26 of SEQ ID NO:30; nucleotide 61 of SEQ ID NO:31; nucleotide 101 of SEQ ID NO:32; nucleotide 201 of SEQ ID NO:33, nucleotide 1466 of SEQ ID NO:34; or nucleotide 75 of SEQ ID NO:35, or a nucleotide sequence complementary to any of the foregoing.

39. A kit, comprising the plurality of nucleic acid molecules of claim 36.

40. The kit of claim 39, further comprising at least one oligonucleotide that selectively hybridizes at or near said nucleotide of a nucleic acid molecule of the plurality.

41. A plurality of nucleic acid molecules, comprising
a) at least one nucleotide sequence of a cytochrome P450 (CYP) gene comprising a single nucleotide polymorphism (SNP), said nucleotide sequence comprising
nucleotides of a CYP2C8 gene, including a nucleotide corresponding to nucleotide 83 of SEQ ID NO:1, nucleotide 251 of SEQ ID NO:2; nucleotide 181 of SEQ ID NO:15, or nucleotide 75 of SEQ ID NO:35;
nucleotides of a CYP3A4 gene, including a nucleotide corresponding to nucleotide 401 of SEQ ID NO:3, nucleotide 437 of SEQ ID NO:4; nucleotide 151 of SEQ ID NO:16, or nucleotide 1466 of SEQ ID NO:34;
nucleotides of a CYP3A7 gene, including a nucleotide corresponding to nucleotide 401 of SEQ ID NO:9, nucleotide 541 of SEQ ID NO:10, nucleotide 151 of SEQ ID NO:17, or nucleotide 201 of SEQ ID NO:21;
nucleotides of a CYP3A5 gene, including a nucleotide corresponding to nucleotide 201 of SEQ ID NO:13, or nucleotide 101 of SEQ ID NO:14;
nucleotides of a CYP2D6 gene, including a nucleotide corresponding to nucleotide 201 of SEQ ID NO:23;
nucleotides of a CYP4B gene, including a nucleotide corresponding to nucleotide 123 of SEQ ID NO:29; or
nucleotides of a CYP2C9 gene, including a nucleotide corresponding to nucleotide 122 of SEQ ID NO:19,
or a nucleotide sequence complementary to any of the foregoing, and
b) at least one nucleotide sequence of a gene comprising a SNP, said gene having a nucleotide sequence comprising
nucleotides of an esterase D (ESD) gene, including a nucleotide corresponding to nucleotide 702 of SEQ ID NO:5, or nucleotide 201 of SEQ ID NO:6;
nucleotides of a glutathione S-transferase (GSTM1) gene, including a nucleotide corresponding to nucleotide 201 of SEQ ID NO:7, or nucleotide 191 of SEQ ID NO:8;
nucleotides of a monoamine oxidase (MAOB) gene, including a nucleotide corresponding to nucleotide 501 of SEQ ID NO:11, or nucleotide 60 of SEQ ID NO:12;
nucleotides of an agouti signaling protein (ASIP) gene, including a nucleotide corresponding to nucleotide 201 of SEQ ID NO:22, nucleotide 26 of SEQ ID NO:30, or nucleotide 101 of SEQ ID NO:32;
nucleotides of a tubulin (TUBB) gene, including a nucleotide corresponding to nucleotide 61 of SEQ ID NO:18;
nucleotides of a tyrosinase-related protein (TYRP) gene, including a nucleotide corresponding to nucleotide 592 of SEQ ID NO:20;
nucleotides of an AIM gene, including nucleotide 201 of SEQ ID NO:24; nucleotides of a GSTT gene, including a nucleotide corresponding to nucleotide 61 of SEQ ID NO:25;
nucleotides of a dopachrome tautomerase (DCT) gene, including a nucleotide corresponding to nucleotide 201 of SEQ ID NO:26, or nucleotide 61 of SEQ ID NO:27;
nucleotides of an oculocutaneous albinism (OCA) gene, including a nucleotide corresponding to nucleotide 135 of SEQ ID NO:28;
nucleotides of a POR gene, including a nucleotide corresponding to nucleotide 61 of SEQ ID NO:31; or
nucleotides of an RAB gene, including a nucleotide corresponding to nucleotide 201 of SEQ ID NO:33,
or a nucleotide sequence complementary to any of the foregoing.

42. The plurality of nucleic acid molecules of claim 41, wherein the nucleotides of the CYP gene comprise
nucleotides of a CYP2C8 gene, including a nucleotide corresponding to nucleotide 83 of SEQ ID NO:1, nucleotide 251 of SEQ ID NO:2; nucleotide 181 of SEQ ID NO:15, or nucleotide 75 of SEQ ID NO:35;
nucleotides of a CYP3A4 gene, including a nucleotide corresponding to nucleotide 401 of SEQ ID NO:3, nucleotide 437 of SEQ ID NO:4; nucleotide 151 of SEQ ID NO:16, or nucleotide 1466 of SEQ ID NO:34;
nucleotides of a CYP3A7 gene, including a nucleotide corresponding to nucleotide 401 of SEQ ID NO:9, nucleotide 541 of SEQ ID NO:10, nucleotide 151 of SEQ ID NO:17, or nucleotide 201 of SEQ ID NO:21; or
nucleotides of a CYP3A5 gene, including a nucleotide corresponding to nucleotide 201 of SEQ ID NO:13,
or a nucleotide sequence complementary to any of the foregoing.

43. The plurality of nucleic acid molecules of claim 41, wherein the nucleotides of gene comprise
nucleotides of an ESD gene, including a nucleotide corresponding to nucleotide 702 of SEQ ID NO:5, or nucleotide 201 of SEQ ID NO:6;
nucleotides of a GSTM1 gene, including a nucleotide corresponding to nucleotide 201 of SEQ ID NO:7, or nucleotide 191 of SEQ ID NO:8;
nucleotides of a MAOB gene, including a nucleotide corresponding to nucleotide 501 of SEQ ID NO:11, or nucleotide 60 of SEQ ID NO:12.

44. The plurality of nucleic acid molecules of claim 41, comprising nucleotide 83 of SEQ ID NO:1, nucleotide 251 of SEQ ID NO:2, nucleotide 401 of SEQ ID NO:3, nucleotide 702 of SEQ ID NO:5, nucleotide 201 of SEQ ID NO:6, nucleotide 201 of SEQ ID NO:7, nucleotide 191 of SEQ ID NO:8, nucleotide 401 of SEQ ID NO:9, nucleotide 541 of SEQ ID NO:10, nucleotide 501 of SEQ ID NO:1, nucleotide 60 of SEQ ID NO:12, nucleotide 201 of SEQ ID NO:13, nucleotide 101 of SEQ ID NO:14, nucleotide 181 of SEQ ID NO:15, nucleotide 151 of SEQ ID NO:16, nucleotide 151 of SEQ ID NO:17, and nucleotide 75 of SEQ ID NO:35; or a nucleotide sequence complementary to any of the foregoing.

45. The plurality of nucleic acid molecules of claim 41, comprising nucleotide 83 of SEQ ID NO:1, nucleotide 251 of SEQ ID NO:2, nucleotide 401 of SEQ ID NO:3, nucleotide 437 of SEQ ID NO:4, nucleotide 702 of SEQ ID NO:5, nucleotide 201 of SEQ ID NO:6, nucleotide 201 of SEQ ID NO:7, nucleotide 191 of SEQ ID NO:8, nucleotide 401 of SEQ ID NO:9, nucleotide 541 of SEQ ID NO:10, nucleotide 501 of SEQ ID NO:11, nucleotide 60 of SEQ ID NO:12, nucleotide 201 of SEQ ID NO:13, nucleotide 101 of SEQ ID NO:14, nucleotide 181 of SEQ ID NO:15, nucleotide 151 of SEQ ID NO:16, nucleotide 151 of SEQ ID NO:17, nucleotide 61 of SEQ ID NO:18, nucleotide 122 of SEQ ID NO:19, nucleotide 26 of SEQ ID NO:30, and nucleotide 75 of SEQ ID NO:35, or a nucleotide sequence complementary to any of the foregoing.

46. The plurality of nucleic acid molecules of claim 41, wherein, when present, nucleotide 83 of SEQ ID NO:1 is A or G; nucleotide 251 of SEQ ID NO:2 is G or A; nucleotide 401 of SEQ ID NO:3 is C or T; nucleotide 437 of SEQ ID NO:4 is T or G; nucleotide 702 of SEQ ID NO:5 is C or T; nucleotide 201 of SEQ ID NO:6 is C or G; nucleotide 201 of SEQ ID NO:7 is C or T; nucleotide 191 of SEQ ID NO:8 is C or T; nucleotide 401 of SEQ ID NO:9 is C or T; nucleotide 541 of SEQ ID NO:10 is G or C; nucleotide 501 of SEQ ID NO:11 is C or T; nucleotide 60 of SEQ ID NO:12 is C or T; nucleotide 201 of SEQ ID NO:13 is A or G; nucleotide 101 of SEQ ID NO:14 is C or T; nucleotide 181 of SEQ ID NO:15 is A or G; nucleotide 151 of SEQ ID NO:16 is C or T; nucleotide 151 of SEQ ID NO:17 is C or T; nucleotide 61 of SEQ ID NO:18 is A or G; nucleotide 122 of SEQ ID NO:19 is A or T; nucleotide 26 of SEQ ID NO:30 is A or G; and nucleotide 75 of SEQ ID NO:35 is A or G, or a nucleotide sequence complementary to any of the foregoing.

47. The plurality of nucleic acid molecules of claim 41, wherein each nucleic acid molecule of the plurality is attached to a solid support.

48. The plurality of nucleic acid molecules of claim 47, wherein the solid support is a glass slide or microchip.

49. A kit, comprising the plurality of nucleic acid molecules of claim 41.

50. A kit, comprising the plurality of nucleic acid molecule of claim 48.

51. The kit of claim 49, further comprising at least one oligonucleotide that selectively hybridizes at or near the single nucleotide polymorphism of a nucleic acid molecule of the plurality.

52. A plurality of oligonucleotides, comprising:
a) at least one oligonucleotide that selectively hybridizes at or near a single nucleotide polymorphism (SNP) of a nucleotide sequence of a cytochrome P450 (CYP) gene comprising the SNP, said nucleotide sequence of the CYP gene comprising
nucleotides of a CYP2C8 gene, including a nucleotide corresponding to nucleotide 83 of SEQ ID NO:1, nucleotide 251 of SEQ ID NO:2; nucleotide 181 of SEQ ID NO:15, or nucleotide 75 of SEQ ID NO:35;
nucleotides of a CYP3A4 gene, including a nucleotide corresponding to nucleotide 401 of SEQ ID NO:3, nucleotide 437 of SEQ ID NO:4; nucleotide 151 of SEQ ID NO:16, or nucleotide 1466 of SEQ ID NO:34;
nucleotides of a CYP3A7 gene, including a nucleotide corresponding to nucleotide 401 of SEQ ID NO:9, nucleotide 541 of SEQ ID NO:10, nucleotide 151 of SEQ ID NO:17, or nucleotide 201 of SEQ ID NO:21; or
nucleotides of a CYP3A5 gene, including a nucleotide corresponding to nucleotide 201 of SEQ ID NO:13, or nucleotide 101 of SEQ ID NO:14;
nucleotides of a CYP2D6 gene, including a nucleotide corresponding to nucleotide 201 of SEQ ID NO:23;
nucleotides of a CYP4B gene, including a nucleotide corresponding to nucleotide 123 of SEQ ID NO:29; or
nucleotides of a CYP2C9 gene, including a nucleotide corresponding to nucleotide 122 of SEQ ID NO:19,
or a nucleotide sequence complementary to any of the foregoing, and
b) at least one oligonucleotide that selectively hybridizes at or near a SNP of a nucleotide sequence of a gene the SNP, said nucleotide sequence comprising
nucleotides of an esterase D (ESD) gene, including a nucleotide corresponding to nucleotide 702 of SEQ ID NO:5, or nucleotide 201 of SEQ ID NO:6;
nucleotides of a glutathione S-transferase (GSTM1) gene, including a nucleotide corresponding to nucleotide 201 of SEQ ID NO:7, or nucleotide 191 of SEQ ID NO:8;
nucleotides of a monoamine oxidase (MAOB) gene, including a nucleotide corresponding to nucleotide 501 of SEQ ID NO:11, or nucleotide 60 of SEQ ID NO:12;
nucleotides of an agouti signaling protein (ASIP) gene, including a nucleotide corresponding to nucleotide 201 of SEQ ID NO:22, nucleotide 26 of SEQ ID NO:30, or nucleotide 101 of SEQ ID NO:32;
nucleotides of a tubulin (TUBB) gene, including a nucleotide corresponding to nucleotide 61 of SEQ ID NO:18;
nucleotides of a tyrosinase-related protein (TYRP) gene, including a nucleotide corresponding to nucleotide 592 of SEQ ID NO:20;
nucleotides of an AIM gene, including nucleotide 201 of SEQ ID NO:24; nucleotides of a GSTT gene, including a nucleotide corresponding to nucleotide 61 of SEQ ID NO:25;
nucleotides of a dopachrome tautomerase (DCT) gene, including a nucleotide corresponding to nucleotide 201 of SEQ ID NO:26, or nucleotide 61 of SEQ ID NO:27;
nucleotides of an oculocutaneous albinism (OCA) gene, including a nucleotide corresponding to nucleotide 135 of SEQ ID NO:28;
nucleotides of a POR gene, including a nucleotide corresponding to nucleotide 61 of SEQ ID NO:31; or
nucleotides of an RAB gene, including a nucleotide corresponding to nucleotide 201 of SEQ ID NO:33,
or a nucleotide sequence complementary to any of the foregoing.

53. The plurality of oligonucleotides of claim 52, wherein the oligonucleotide comprises a probe.

54. The plurality of oligonucleotides of claim 52, wherein the oligonucleotide comprises a primer.

55. The plurality of oligonucleotides of claim 52, wherein the oligonucleotide comprises a primer of an amplification primer pair.

56. The plurality of oligonucleotides of claim 52, wherein each oligonucleotide of the plurality is attached to a solid support.

57. The plurality of oligonucleotides of claim 56, wherein the solid support is a glass slide or microchip.

58. A kit, comprising the plurality of oligonucleotides of claim 52.

59. A plurality of oligonucleotides, comprising at least one oligonucleotide that selectively hybridizes to a nucleotide sequence as set forth in
SEQ ID NO:1, said nucleotide sequence comprising nucleotide 83 of SEQ ID NO:1, wherein nucleotide 83 is G;
SEQ ID NO:3, said nucleotide sequence comprising nucleotide 401 of SEQ ID NO:3, wherein nucleotide 401 is T;
SEQ ID NO:4, said nucleotide sequence comprising nucleotide 437 of SEQ ID NO:4, wherein nucleotide 437 is G; or
SEQ ID NO:8, said nucleotide sequence comprising nucleotide 191 of SEQ ID NO:8, wherein nucleotide 191 is T,
or to a nucleotide sequence complementary thereto.

60. The plurality of oligonucleotides of claim 59, further comprising at least one oligonucleotide that selectively hybridizes at or near a nucleotide sequence as set forth in
SEQ ID NO:1, said nucleotide sequence comprising nucleotide 83 of SEQ ID NO:1, wherein nucleotide 83 is A;
SEQ ID NO:3, said nucleotide sequence comprising nucleotide 401 of SEQ ID NO:3, wherein nucleotide 401 is C;
SEQ ID NO:4, said nucleotide sequence comprising nucleotide 437 of SEQ ID NO:4, wherein nucleotide 437 is T; or
SEQ ID NO:8, said nucleotide sequence comprising nucleotide 191 of SEQ ID NO:8, wherein nucleotide 191 is C,
or a nucleotide sequence complementary thereto.

61. The plurality of oligonucleotides of claim 59, further comprising at least one oligonucleotide that selectively hybridizes at or near nucleotide 251 of SEQ ID NO:2; nucleotide 702 of SEQ ID NO:5; nucleotide 201 of SEQ ID NO:6; nucleotide 201 of SEQ ID NO:7; nucleotide 191 of SEQ ID NO:8; nucleotide 401 of SEQ ID NO:9; nucleotide 541 of SEQ ID NO:10; nucleotide 501 of SEQ ID NO:11; nucleotide 60 of SEQ ID NO:12; nucleotide 201 of SEQ ID NO:13; nucleotide 101 of SEQ ID NO:14; nucleotide 181 of SEQ ID NO:15; nucleotide 151 of SEQ ID NO:16; nucleotide 151 of SEQ ID NO:17; nucleotide 61 of SEQ ID NO:18; nucleotide 122 of SEQ ID NO:19; nucleotide 592 of SEQ ID NO:20; nucleotide 201 of SEQ ID NO:21; nucleotide 201 of SEQ ID NO:22; nucleotide 201 of SEQ ID NO:23; nucleotide 201 of SEQ ID NO:24; nucleotide 61 of SEQ ID NO:25; nucleotide 201 of SEQ ID NO:26, or nucleotide 61 of SEQ ID NO:27; nucleotide 135 of SEQ ID NO:28; nucleotide 123 of SEQ ID NO:29; nucleotide 26 of SEQ ID NO:30; nucleotide 61 of SEQ ID NO:31; nucleotide 101 of SEQ ID NO:32; nucleotide 201 of SEQ ID NO:33, nucleotide 1466 of SEQ ID NO:34; or nucleotide 75 of SEQ ID NO:35,
or a nucleotide sequence complementary thereto.

62. A kit, comprising the plurality of oligonucleotides of claim 59.


SEQUENCE LISTING (35 sequences)

SEQ ID NO: 1 - DNA, 280 bp, Homo sapiens CYP2C8E8_92 (265)
  misc_feature (83)..(83): n is r
ttcttataatcagattatctgttttgttacttccagggcacaaccataatggcattactgacttccgtgctacatgatgacanagaatttcctaatccaaatatctttgaccctggccactttctagataagaatggcaactttaagaaaagtgactacttcatgcctttctcagcaggtaatagaaactcgtttccatttgtatttaaaggaaagagagaactttttggaattagttggaatttacatggcacctcctctggggctggtagaattgcta

SEQ ID NO: 2 - DNA, 612 bp, Homo sapiens CYP2C8_RS947172 (371)
  misc_feature (251)..(251): n is r
ttattcggatttttttcttgctgttttgagtttcttgtagactctggaaaatagtcctttgttgaaggtatattttgcaaatattttctcccattctgtaggttgtatgtttactctgcttgtcatttcttttactgtgcagaagctctttagtttaattaggtcccattgtcaactgtttttgttgaaattgcttttaaacattgagtcataaatccttagcctacaccaatgctcagaagagttttttntaggttttttctagaatttttatgatttcaagtctcatatttaagtctttagtccatcttgagttaatttttgtatgtggtgagatataagaatcatatttcattcttctacatgttcccctgggtaatatcagccaagcacaaatcccacagctaccagcgtaggtggctctttcctgcaagaaccacctcctagctggaagccaataggcacagcctattacaacatctgctggcaaaataacatagcatttgggaaggagaaaacttttatcgtatctcagctaacaccatacccacatcaccccagctaatcggaaggtcttgagtgtgttcacaaacccaatacattgctagtaca

SEQ ID NO: 3 - DNA, 770 bp, Homo sapiens CYP3A4E7_243 (664803)
  misc_feature (401)..(401): n is k
tcagttcagtgtctccatcacacccagcgtagggccagctgcatcactggcacctgataacaccttctgatggagtgtgatagaaggtgatctagtagatctgaaagtctgtggctgtttgtctgtcttgactggacatgtgggtttcctgttgcatgcatagaggaaggakggtaaaaaggtgctgattttaattttccacatctttctccactcagcgtctttggggcctacagcatggatgtgatcactagcacatcatttggagtgaacatygactctctcaacaatccacaagacccctttgtggaaaacaccaagaagcttttaagatttgattttttggatccattctttctctcaataagtatgtggactactatttcctttaatttatcttnctctcttaaaaataactgctttattgagatataaatcaccatgtaattcakccacttwaaatatacagttcagtgatttgtagtacatttgaagatatgtgtgaccatcatcattttaaactttaaaactttttttgtcaatctagagacctcatacatttttagctatcagccccctgtcacaaaccctgtcatcatatgcaaccactaatcaactttctgcttctatggatttgcctattctggacacttcatagaaatgatattaattcatcagggttttttattctctagttcatgaatttgtactttagtctgtatcattttctttcttctgctggcttcaggcttagtttgcccttcttcgtt

SEQ ID NO: 4 - DNA, 910 bp, Homo sapiens CYP3A4E12_76 (869772)
  misc_feature (437)..(437): n is y
gaggagatgagaaggaaagtgcaaaataacaattttgaaatagtacaagacaatttccccttctccttcctcatgaccaatgtaagtgtgacttgaggcaggaacctacttttccatcagtcagtcccatcacttatgtgccttttatagtgtggacacatcaccaccctgaatataatttcagtgtttagaaataagtattctttgcaacactatttatctcatctcaacaagactgaaagctcctatagtgtcaggagagtagaaaggatctgtagcttacaattctcatagcaaaataagcatagcaggatttcaatgaccagcccacaaaagtatcctgtgtactactagttgaggggtggcccckaagtaagaaaccctaacatgtaactcttaggggtattatgtcattaactttttaaaaatctaccaangtggaaccagattcrgcaagaagaacaaggacaacatagatccttacatatacacaccctttggaagtggacccagaaactgcattggcatgaggtttgctctcatgaacatgaaacttgctctaatcagagtccttcagaacttctccttcaaaccttgtaaagaaacacaggttagtcaattttctataaaaataatgttgtattaataattcttttaactgagtggtctgtattttttaaaaagaatatgcttgtttaatcttttactaatttgttctctgggccaaagaatcaattaggcccatctgtgatctttaagggtgcttcagttctggagttcaaaagctgtagcatcaaaaacatcatgtaaagcccatgtagattagcatggcataattatctgcagtctccttgaacttgagcaaagttacattcatttcaagtcgattggaaagatggggaagtatttt

SEQ ID NO: 5 - DNA, 989 bp, Homo sapiens ESD1216961 (677)
  misc_feature (702)..(702): n is y
cagagtaaacacctatctttttatcaaagttatgtaagtacatagtttaaagtaaactgtaactaaatagtaaatacttccacacactgttatgaaaaactaaatatatataaaaactaaatatataaaaaactctccaaccttctcattttccatattccagagataattaatttacctcttttagttgatttttctggtatttatgtccctatctacataatatgactttattactgcttctagatttttcagtttttagttattatctattggcttcccattatagaaggattttagttaactttcagctccctgtctttgctctctgcatgcccctacatttctccttccactatcgtatcacatataattttggttaatcaatattaagaagttataaatgctattcagagataagccatgtagtatattagcattactttcctttttctttcagtgttttttgttttcattgagctggtaattctctcctttttgttactggttatccttccttgttaggaatagactcccacaaaggccccatcatgggaagctttctgtatgctcctttccattctctgcatattggtgattttctttttccacattattctaagcaacttttcttgttgatgactgacggcacaaaaaatttctgacatttgtttttaggttttcaacttttgttagggaagntgaaacatttactttcattataatactatatcaaatccatattcaatgcagggatatctgttatcaatacgtaaaatcaagagaacataatcttgttacagaatggttggtgccttaagacctttcttgcacacttaaacatttgttaagagggtatatttcatgttgttttgttattacaacaaaatttttaaaagaggacctttctcaagcagttgtggaataaatcaggtctaaagtatgttcaacagtggtatgattaacacttatgtaaaagcaaaaaaaaa

SEQ ID NO: 6 - DNA, 401 bp, Homo sapiens ESD1216956 (648)
  misc_feature (201)..(201): n is s
ctggaattacagccataaagaagagacataaaaaaaaagattatctgaaatagagaatatttggaaaaagatcatggagttagaagaagcttctagtttccaatttgtggaaggagttacaaaggaaaaaaaatcatgttcttgctggagccaagaggagattaagaatattaaaggtttagaaggaaaaaggccattttnttaagaatccctgagatatgggattcttgccactagtctaggactgcttgaaagtagggaagagactattattagaaaatagaattccgaagaccttgtaaataaaagcctggcaagtcagattttgaaccaacttaaaagcatatttctgatttaatcagcctaagagcttaaataactttaaatttctgtttggtact

SEQ ID NO: 7 - DNA, 401 bp, Homo sapiens GSTM1421547 (527)
  misc_feature (201)..(201): n is y
gtgttcttcagtatgagacggtggctccagtggcctttgaagtcacaccgtgatatgtgacccatggtacaacctccacgagaacaatgtccaacctgccaactttcttctttcaaggtagaaggaagactttcaaaagagttgtgcaatggattagcctggggttgactgctttaaaggatattgcaaataataatgganatatggaaatagatgatagacctttaatgagaaatcattttgcaatgtaaaccaggctgttgtgctgcaaaaaaagtagtttttttgttttgttttgttttgttttgttttgttttgttttttgtaaattagctaaaacattgttaggactccagaggatgaacccagtatatcaaaaaagtttcaaaccacctggataa

SEQ ID NO: 8 - DNA, 373 bp, Homo sapiens GSTM1E2E4_240 (200)
  misc_feature (190)..(190): n is y
ctcactgggggacactcacaaattctggattgtagcagatcatgcccagctgcatatggttgtccatggtctggttctccaaaatgtccacacgaatcttctcctcttctgtctccccacctgccacccacaaaacacagactcaatctcagcatcacagcccagccaaggaggaggccaccttcccccnacacattgcagccacccacactcacacaggttgtgcttgcgggcaatgtagcacaagatggcgttgctctgggtgatcttgtgagccccatcaatcaagtagggcagctggatggatgggaaaccggcgaagctcagttgggccaccaggctccccagcatccccttcccataagcaagagca

SEQ ID NO: 9 - DNA, 751 bp, Homo sapiens CYP3A7RS2037498 (121)
  misc_feature (401)..(401): n is r
cactcggtgctagaagcaaaaagagaaattttattgacagaaagtgactcgtgaagtcagaagttaatcagaagtgcagtcctcaacctcccttctgagaatatagccccttggagaccagaaatctgatgggtgttgcctgagtcaccagtgaaaaggaatatccatctaaatccttggaaagcaggatgttttcctgaaacaaatctggctaaaatgttcaattgctttttggtcttttccctggtcctaactgcatacccatgaagaagtcctgggccttttattcttctgtcttcttcatcaccaatgtccacaaagattattctcctgacatttccagaacttgtcagcagctttggtgggagcactttcaaatactgcaacattccccaaatctntacaatcttcagtgacacctgggttaccccttcttgcccccctgacctgcagatatcttttggctggactccagggctcatgaaggcaaagctgagttagccagccttgcagcctcaattcccagagaagacctctgttcactcgaggctgtcagagggctcaaatgtaacacacactacacttcagcaggtggcctgataggaccttttgttggagaatgtgtaaaataatagccttgtacttgaaatgactctttaccaatctatgatctagagaaagcataataccaataaaatgtatttccaaacagtaatgtttatcctttgacaatttattgaagggaagtgct

SEQ ID NO: 10 - DNA, 614 bp, Homo sapiens CYP3A7RS2222411 (113)
  misc_feature (541)..(541): n is s
gaacacagaaataaatatagtgcctgttccaaccatgtaactcaccaccaaaaccaatacgaaaggagtgtgagaaatgtcaacagagctgtgatttaccagtgaagatttctgtgatgcagttgtgggtaaattcaactatctaataattgtattctgttaccatgaacctgatgatactgatgtgaaaccaaactataatctcaaagtttagagctctgctgtccaatgtggtagccactattgacatgtggctatttaaaatcaaattttattcacttaaacttacatacaacttacaatttttcatcagtggcactagcacatgtgcagtgctcaatagctatatgtgtctagtgatttctagatgaaacactaaaacagtaggctatttcctttatcacagcaagttgtattgggatagatagagagatgatagctagataatcagtaagtagataaatagatgattgatggacagatgatagatagatagacagacagacagaagtagatgtagacatggacatatttaaatctntatgccaaagtcagcctactcctgctctctagaaccaatgattaaatattcaggaattttgtgacatgattgc

SEQ ID NO: 11 - DNA, 565 bp, Homo sapiens MAOB1799836 (465)
  misc_feature (501)..(501): n is r
gtaggtcaaagatatctgagagcctggagaccaactctccagtaacccccatcttaagacccataaaatactattttaactattgtctcaggtgaagtgaacaccttagcccgaagtaataatgaaaatacctaacatgtcacccgaagtaaaaagagcccatggggcaaacgatcaacctgactatacttgaaaggtaacctaggtctgatattctagaaccaaacatgcttctattaggactcaaaattcaattcatttcacaggaaagattttctgacagttcctctgatgtcatccctatttgaggtgtgttcataactattaaaatttatggtaagcctaatgattggaacctcttataccacaggagaaagaccttttggcgcctcttaaaggttgtatggagtgttctggcctttaccttggtgtttttgatggatatctcaagagacagttacttagtcctttagggagcagattagaagaaagatggtgtcncttttgctatttgccagtgtgttctttcatttcagatcctgcatgccatggggaagattccaga

SEQ ID NO: 12 - DNA, 160 bp, Homo sapiens MAOB6324 (477)
  misc_feature (60)..(60): n is y
tgaacctaggatgtccctgcacagcccatcaccaccacctttttggagagacatttgccntccgtgccaggcctgctcaggctgattggattgaccaccatcttttcagcaacggctcttggcttcctggcccacaaaagggggctacttgtgagagtct

SEQ ID NO: 13 - DNA, 401 bp, Homo sapiens CYP3A5776746 (537)
  misc_feature (201)..(201): n is r
agtccttgtgagcacttgatgatttacctgccttcaatttttcactgacctaatattctttttgataatgaagtattttaaacatataaaacattatggagagtggcataggagatacccacgtatgtaccacccagcttaacgaatgctctactgtcatttctaaccataatctctttaaagagctcttttgtctttcantatctcttccctgtttggaccacattacccttcatcatatgaagccttgggtggctcctgtgtgagactcttgctgtgtgtcacaccctaatgaactagaacctaaggttgctgtgtgtcgtacaactaggggtatggattacataacataatgatcaaagtctggcttcctgggtgtggctccagctgcagaatcgggc

SEQ ID NO: 14 - DNA, 198 bp, Homo sapiens CYP3A5RS15524 (247)
  misc_feature (101)..(101): n is y
aaattagacacgcaaggacttcttcaaccagaaaaacccattgttctaaaggtggattcaagagatggaaccctaagtggagaatgagttattctaagganttctactttggtcttcaagaaagctgtgccccagaacaccagagatttcaacttagtcaataaaaccttgaaataaagatgggcttaatctaatgta

SEQ ID NO: 15 - DNA, 381 bp, Homo sapiens CYP2C8_RS1891070_ (357)
  misc_feature (181)..(181): n is r
gttgtatgtttactctgcttgtcatttcttttactgtgcagaagctctttagtttaattaggtcccattgtcaactgtttttgttgaaattgcttttaaacattgagtcataaatccttagcctacaccaatgctcagaagagttttttataggttttttctagaatttttatgatttcangtctcatatttaagtctttagtccatcttgagttaatttttgtatgtggtgagatataagaatcatatttcattcttctacatgttcccctgggtaatatcagccaagcacaaatcccacagctaccagcgtaggtggctctttcctgcaagaaccacctcctagctggaagccaataggcacagcctattacaacatct

SEQ ID NO: 16 - DNA, 301 bp, Homo sapiens CYP3A4_RS2687117 (232)
  misc_feature (151)..(151): n is y
cttcaggcttagtttgcccttcttcgtttactatgttgtggcatgaacatagattactgatttgtgatttttttgttcctctaaatttagacattacagctgtaactttccctctgagcacttcctttgctaaatcccatgagattgtggnctatcacatcttagtttttgttcacctcaaaacagtttctatttgcccttttggtttctactttgactcattggttacttaaatgtttattatttaacttccacatatgtgtgagtttctcaattttctttcccttattgattttatctt

SEQ ID NO: 17 - DNA, 301 bp, Homo sapiens CYP3A7RS2687075 (229)
  misc_feature (151)..(151): n is y
cttgggttttaattagaaacttaaagaatgaatcaggtggggacaggtagaaagtaagatcagagttcctttccgaggagtagtctgctgaatttgagcttcctaaaaatagtctttttatgtacagaaaacacatcataaaattcattanacaatgtcacttattgttccatgccaggcaaagtcatgtccttctgggacttatgtctgcacatttaactatgggtggtgttgtgttttgtgcttagatggtccctatcattgcccagtatggagatgtgttggtgagaaatctgaggcg

SEQ ID NO: 18 - DNA, 121 bp, Homo sapiens TUBB42302897_GA (761)
  misc_feature (61)..(61): n is r
ggggtcgatgccatgctcatcactgatgacttcccagaactgcaaacaaagtgggagaggnggggtcggcaccggcccatagccccagccaggccctttcccttgtgaagccttttagccc

SEQ ID NO: 19 - DNA, 422 bp, Homo sapiens CYP2C9RS1934969_TA (39)
  misc_feature (122)..(122): n is w
gtccattcatttttcagttgcctatacatccatccattcatccatttatccatccactcatccatccattcattcatgcatgcacccatccacccatctatctcttcatctcttctacgatncactgaacagttattgcatattctgtttgtgccagttacagagacagtgtttgtcactgtcacagttacgcatgaggagtaactgctctctgtgtttgctattttcaggaaaacggatttgtgtgggagaagccctggccggcatggagctgtttttattcctgacctccattttacagaactttaacctgaaatctctggttgacccaaagaaccttgacaccactccagttgtcaatggatttgcctctgtgccgcccttctaccagctgtgcttcattcctgtctgaagaagagcag

SEQ ID NO: 20 - DNA, 1190 bp, Homo sapiens TYRP1E6_354 (886938)
  misc_feature (592)..(592): n is y
tccaagacaaccagagcattcttctcccaattgtgtcagcagtttttagcagaaactggagattttcactatctttattcccatgtagaacaatgtctgccttgtcaaatagagtgtctatatattttgactggtatcctttgcaggtcattatttttaacctggaggctacatttagaatgtcatgccactagcataaaaccagcctagctctgggtatagcaaacaaatgtcatcaaccataggtacagagaagcagtgctgttatattaaggcaaaaattggcctgacgagccttgaaaaaattgtttcccaatataatcattgctattacctggaaaagtgaaatatttgcaatagttttactattctcctccttaccatgtgtctaggttacagtgaccccacgggaaagtatgaccctgctgttcgaagtcttcacaatttggctcatctattcctgaatggaacagggggacaaacccatttgtctccaaatgatcctatttttgtcctcctgcacaccttcacagatgcagtctttgatgaatggctgaggagatacaatgctggtaagacattttcatatgcnttttgcatgctcagctgggcrgattgtttagatggcatagttatcagttcaagctgagcactcagcgcataaaaacactttcaaaataaggatagcatagctgtaatatcaagtcacttccagacattcaattctactttgaaaatgcaggcaagaagtctctccaaatagttattataggatgatatttttaaccatttgctgggactaaccaaatcatctctcctaatttttgctacgaaaaaatggctactatgtttcacaaattgttcttaccctcatcttccaacataaattctcattacttttaaatataaatgaggaaaaccttatagagctcttctttgatatcccaaaagactaaaaattgtatacccataatccacagttcttatttttattacttaatggcccctcacaaatagaaattgtgttccacttgaaaagttaatgttggcttttatatcacactataggaaatctcttttaagttcatttcaatgcattctttaaaatactatttaaaaggcctctttctctgccttcctatgcagctgagaggctagcattatttttcaaacaataaaagtgcttccac

SEQ ID NO: 21 - DNA, 401 bp, Homo sapiens CYP3A7RS2687145_TC (140)
  misc_feature (201)..(201): n is y
acatctttgtctatacatttctgctgaatttgaagagatgctgaatattcttaaaccattgtgttccctggtgggctgatggactgtgattttataaggtggcctcagccaactgcagcagctgttccctgtcagaggggctagaggtttggcaagagcggtggaagaggtgcagtggtgtgttcgttcactagaagcatnagggagaaggttttgcctgtttgtatttcatcttctctcatcaagtcctcagaaaccacagtgctgtctgcagggtgctgtggatctggcatggcccatacaggcaacatgactgagtagaaaggacacacagctctggatgtccttgggccccacagcaactgcccttgaaacatttagtccttgtgagcatttgatga

SEQ ID NO: 22 - DNA, 401 bp, Homo sapiens ASIP81936_GA (559)
  misc_feature (201)..(201): n is r
tgccaccagtctaataagcagcttagcatatggtagaggctctgaaaggcctgaagttaagacacttggtgaactttgtttaatttagcatttctgaaacttaatgaatcacagaactcctgtcaacagtaacaaacttcaggaaatgctccagaacatatgcaagtctgggatggaccagtcctgtcatgtcagggttgngaatgaaggctcaggggaaaatatgaggggcactggagcctggcattggagatctggtttgacttcacctgataataatcatagacatactgtgtggtaggcactgtgaggtgagtatggtctttattcatatttcacagttgaggaacttgaggcttaggagaattaagtaactagcacggatcacacagtttttaact

SEQ ID NO: 23 - DNA, 401 bp, Homo sapiens CYP2D6_RS769261_GT (423)
  misc_feature (201)..(201): n is k
gggcctctcggcaattttggtgactcttgcaaggtcatacctgggtgacgcatccaaactgagttcctccatcacagaaggtgtgacccccacccccgccccaggatcaggaggctgggtctcctccttccacctgctcactcctggtagccccgggggtcgtccaaggttcaaataggactaggacctgtagtctggggngatcctggcttgacaagaggccctgaccctccctctgcagttgcggcgccgcttcggggacgtgttcagcctgcagctggcctggacgccggtggtcgtgctcaatgggctggcggccgtgcgcgaggcgatggtgacccgcggcgaggacacggccgaccgcccgcctgcgcccatctaccaggtcctgggcttcgggc

SEQ ID NO: 24 - DNA, 401 bp, Homo sapiens AIM35397_GT (928)
  misc_feature (201)..(201): n is k
tataattaggagaactgcatacatttttcaggccagacgtttcacttaactcaatgtatacagagcttggagatatccatacacagtcgcaagtacaatgcagcctgtgcaatgccctggagttatagcacagccacctggacttacataaagacaatagcagaaatccctttgttcagtgtcttcacagctgcaacttangtaagtggaggttaagaggctcagaaaagctggcacctgggaaggaaagccagctgtccccaatcctggtgtggtgctcctataacccacccagttacacatctgagctaaggtcattttgcatgctgactttgtcacctgtcatattaatattgcttctcttctgtatcatctacaaatctgataagcctatctatgaa

SEQ ID NO: 25 - DNA, 121 bp, Homo sapiens GSTT22267047_TC (464)
  misc_feature (61)..(61): n is y
tggccttggagggatcacagcctctctgaaccttagcttgccttctgaaaaggaggataangttaccttctgctctgtagggatggaaagaaaatactgaatggagttgacagagttcttg

SEQ ID NO: 26 - DNA, 301 bp, Homo sapiens DCT2892681_GC (650)
  misc_feature (201)..(201): n is s
atactgtctgagttcaagtcagggttcaaattgtgatgacagaaagagacaagagactatcaggacacaaggaagatgcagaatcagtgctgtaagaggcaaataacttatagaaagtatcagaattcaccaagggaaaaatgtaaataataaaggagaaggcaagatcctaaggcaattagtaatctggagagataaaanaataggggaaaataataaggaggaattgaaattgtagctctcaggagggctataaaacatacaataaatgctctagtaaggctgcacacagtggctcatg

SEQ ID NO: 27 - DNA, 121 bp, Homo sapiens DCT2296498_GA (701)
  misc_feature (61)..(61): n is r
gcccaaatcaactcatatagagtgactatgatggcgaggatcaagatttcgggaagaaaancagttaagttttcaacgatgtatgaatctctctctccaagcaggactataaacccctttg

SEQ ID NO: 28 - DNA, 435 bp, Homo sapiens OCA_8(217458,719) (217458)
  misc_feature (135)..(135): n is y (c/t)
gatcgacccacctcggaaagtgctgggattacaggcgtgagccaccatgcctgggctgccatttcatttccccttgtttatttccagggcctggactttgccggattcactgcacacatgttcattgggatttgncttgttctcctggtctgctttccgctcctcagactcctttactggaacagaaagctttataacaaggaacccagtgagattgttggtgagtacaagtgcaacctcatgtaggctcagatttcatgaccataatattgtttgtttaccaggagaagttcttattaggaagtatctgttgatgggttgctggatgctcaataccagtgactctccacgtccaccttctagtatacactgttttcagggctgctatcatgagctgtgcctctttagttttcgtgaagtgtactgtccctaaaa

SEQ ID NO: 29 - DNA, 273 bp, Homo sapiens CYP4B1RS751028_GA (292)
  misc_feature (123)..(123): n is y
cgaagtcatcaaaggagagccataagactggattgtaaacaggaggaatgaatttctatgtctcaattttaaaatgtgggcatctgagacccaggaagaggaggctcagtggtgggcagcacntgtcagtgtctgggtatgggatcctcgagtactggttttggagaattccgtggcagagtctcctcccatgacctgctacatctggttctttttgctctggtttgctgatttcaatgtaaaaatcacttggtgggcctgcagggaggaggg

SEQ ID NO: 30 - DNA, 42 bp, Homo sapiens ASIP8818 (859)
  misc_feature (26)..(26): n is r (a/g)
agcgcccccactcccggccgcgagcnggcagggcttcgggga

SEQ ID NO: 31 - DNA, 121 bp, Homo sapiens POR17685_GA (691)
  misc_feature (61)..(61): n is y
acagaactttattccaagggccagaggttatttaaaattatttacactcactggaaatcangtggaggggctgggcccagctcagcagagagaaagagcccttctgtgcagccaccgaggg

SEQ ID NO: 32 - DNA, 201 bp, Homo sapiens ASIP2424987_GA (861)
  misc_feature (101)..(101): n is r
tgagccaagatcacaccactgcaccctagcctgggcgacagagcaagactgtctcaaaaaaaaacaaaagcggagggggggaagttaaaacattgttagtnaattctgggtgaatggatggtattgcctaccaaaggctctaaatttgaggcctaaatctctgtgttaaaaggcataatggccaggcacggtggctcacgc

SEQ ID NO: 33 - DNA, 401 bp, Homo sapiens RAB272444039_GT (925)
  misc_feature (201)..(201): n is k (t/g))
gggagttgggggcaggcaggttttcaaaataacagatctttcaaggtttgggcatggtgttgagggcgacagccttcttatctgagcttgcagtccagttagtgaaagcctggagttagtggaattaccccatctttcagcagaaaatcattctagtctataccagactatactcccacctccatcctgcaagtccatacntggctttctcatcactacttatcactactaggtgaagacaatttaatgttaagtctcaattgatggtatattttcatataaacaatttttgttctcttatttattattcaagtcctgagtagtattcctagggcctgttttccagaaagaaaatactccactcctcaaatacagaaatctaataacgtaaatgatttttg

SEQ ID NO: 34 - DNA, 2726 bp, Homo sapiens CYP3A4E7_117 (664802)
  misc_feature (1466)..(1466): n is y
ctgggactagagtctgcacatttaactatgggtggtgttgtgttttgtgcttagatggtccctatcattgcccagtatggagatgtgttggtgagaaatctgaggcgggaagcagagacaggcaagcctgtcaccttgaaagagtaagtagaagcgcagccatggggttctgagctgtcatgaacccctccagctgcctgccatggagctgatattcctgctgttgggttattccagtgaccagacaaaaggagggctgtggtaatgcaacttcaatgggtctcccaagatggggcagctccgatgaggaggtggggcagctggaggaaaaggatcttctcccctgtgcacaggggccagggtttacatatccattaaattgtcaccttggatattctagaagactaaatatatcctttagggggaaaaagtgtgattgtaccaaagttttaagcatggagtgtatgggatggtggaaggggaaggcacttggtatctgttggttggcagtgagtaggttgggagagttataatggagaacttagaataactttgatcatttcatgtttttttctgaggatatcagtagaatactaaatattaaaattcctaccatttctttttcctccagtctcaaagagagagggtggtaaaaacactataggtagggcaagcctattatttgctatctacacttatgcagtaaaaacaggtgtaatctgagtttgtcctgggcagaccagggatatgtggtcactcactatagaaatttccaaatcaaattttgagagatttttttttaaccaggacattattggtcattatattttacaaaaataattctgctgtcagggcaacctcagctcaccacagctggggatagtggaattttccaaagcttgagcagggagtatagagaataaggatgatatttctaggagctcagaacagggtactgttgctttgtaaagtgctgaagaggaatcggctctgggcatagagtctgcagtcaggcaatatcacctgtcttgagccccttaggaagagttaattattctactcttgttctgctgaagcacagtgcttacccatcttgtatcatccacaatcaatacatgctactgtagttgtctgatagtgggtctctgtcttcctatgatgggctccttgatctcagaggtaggtctaattcagttcagtgtctccatcacacccagcgtagggccagctgcatcactggcacctgataacaccttctgatggagtgtgatagaaggtgatctagtagatctgaaagtctgtggctgtttgtctgtcttgactggacatgtgggtttcctgttgcatgcatagaggaaggakggtaaaaaggtgctgattttaattttccacatctttctccactcagcgtctttggggcctacagcatggatgtgatcactagcacatcatttggagtgaacatnctctcaacaatccacaagacccctttgtggaaaacaccaagaagcttttaagatttgattttttggatccattctttctctcaataagtatgtggactactatttcctttaatttatcttkctctcttaaaaataactgctttattgagatataaatcaccatgtaattcakccacttwaaatatacagttcagtgatttgtagtacatttgaagatatgtgtgaccatcatcattttaaactttaaaactttttttgtcaatctagagacctcatacatttttagctatcagccccctgtcacaaaccctgtcatcatatgcaaccactaatcaactttctgcttctatggatttgcctattctggacacttcatagaaatgatattaattcatcagggttttttattctctagttcatgaatttgtactttagtctgtatcattttctttcttctgctggcttcaggcttagtttgcccttcttcgtttactatgttgtggcatgaacatagattactgatttgtgatttttttgttcctctaaatttagacattacagctgtaactttccctctgagcacttcctttgctaaatcccatgagattgtggcctatcacatcttagttttgttcacctcaaaacagtttctatttgccctttgggtttctactttgactcattgggtacttaaatgtttattatttaacttccacatatgtgtgagtttctcaattttctttcccttattgattttatctttattccatgataggtgacagagatatgctgtgttatttctatcttgactacctactatttcttgaacagcaagattaattttgagcttcagattatgatttgggttattctaggagactgtagtccaatagataaaggcaaagagattagggcattgaattttgttccttttatccttcaaaagatgcacaaggggctgctgatctcactgctgtagcggtgctccttatgcatagacctgcccttgctcagccactggcctgaaagaggggcaaaagtcatagaaggaatggcttccagttgagaaccttgatgtcttttactcttctggttggtagagaaaactagaattgctccaggtaaattttgcacattcacaatgaatttctttttctgtttttgttttgtttttcctacagcagtctttccattcctcatcccaattcttgaagtattaaatatctgtgtgtttccaagagaagttacaaattttttaag

SEQ ID NO: 35 - DNA, 631 bp, Homo sapiens CYP2C8_RS1891071 (369)
  misc_feature (75)..(75): n is r
taatctgtctaaatttgatgacacaatttaaaatgacatctttgtacaatggaggaggatgacagagatcagtanaaacagtatggcagtagcaaaataagtaaagcactgatgaagtgtctggatttcagcaaaggtaatttgtggtaaggagagccagcataaattgccctagtattgaatgttggttttattatgaaaagtccactttgaacagtaggttcatttctcattttaaaaattccatgctctaatgctgtggtggggagatgaaaacaatctttattgaagcataagtggaaattctagaattgtactgaggcatcctgacataaattccagtctgggaagtaatctaaaagttagtctcttacaaaggtgtttctatttaagcagaggccatacctaaaggaattttattattctaggagtgtgtttcataaaaatgctatttgaccaaatagggacatttggaagggggtttaataattgcatctttcacatacaacttttctttagaatttacaatttacccttagagtaaactctacatttccttagaatttacatttaaatagttcctgctttgcagcagataacatacctttttccctgtgtcaggatcccactg